(19) United States
(12) Patent Application Publication
(10) Pub. No.: US 2023/0233596 A1
(43) Pub. Date: Jul. 27, 2023

(54) BETA-GLUCAN FOR IMMUNO-ENHANCEMENT AND/OR IMMUNO-BALANCING, AND FOR ADJUVANT USE

(71) Applicants: GN Corporation Co Ltd, Yamanashi (JP); Sophy Inc., Kochi (JP)

(72) Inventors: Takashi ONAKA, Okayama (JP); Samuel JK ABRAHAM, Yamanashi (JP)

(21) Appl. No.: 18/001,994

(22) PCT Filed: Jun. 15, 2021
(86) PCT No.: PCT/JP2021/022709

§ 371 Date: Dec. 15, 2022

(30) Foreign Application Priority Data
Jun. 16, 2020 (JP) 2020-104116
Aug. 13, 2020 (JP) 2020-136861
Mar. 25, 2021 (JP) 2021-051608

(51) Int. Cl. A61K 31/716; A61P 35/00; A61P 31/14; A61K 9/00

(52) U.S. Cl. CPC A61K 31/716; A61P 35/00; A61P 31/14; A61K 9/0053

(57) ABSTRACT

Methods for inducing, enhancing and/or balancing an immune response are provided. The methods include administering a beta-glucan produced by Aureobasidium pullulans AFO-202 (FERM BP-19327). The methods include administering a beta-glucan produced by Aureobasidium pullulans AFO-202 (FERM BP-19327) as a vaccine adjuvant.

### RELATED APPLICATIONS

This application claims the benefit of the filing dates of Japanese Application No. 2020-104116, entitled “TREATMENT OR PREVENTION AGENT FOR VIRAL INFECTION”, filed Jun. 16, 2020; Japanese Application No. 2020-136861, entitled “BETA-GLUCAN-WIDE-SPECTRUM IMMUNE-BALANCING FOOD-SUPPLEMENT-BASED ENTERIC (B-WIFE) VACCINE APPROACH TO COVI”, filed Aug. 13, 2020; and Japanese Application No. 2021-51608, entitled “BETA-GLUCAN VACCINE ADJUVANT”, filed Mar. 25, 2021; the contents of each of which are incorporated herein by reference in their entirety.

### TECHNICAL FIELD

The present invention relates to Beta-glucan for immuno-enhancement and/or immuno-balancing, and for adjuvant use.

The present invention also relates to a composition for regulating cytokine production, regulating immune cells or inhibiting blood coagulation, which can treat or prevent viral infection, especially SARS-CoV-2.

The present invention also relates to Beta-glucan Vaccine Adjuvant for conquering cancer through immune-enhancement and/or for tackling cancer in specific immunocompromised populations.

### BACKGROUND ART

Cancer is a deadly disease and, as the second leading cause of death globally, causes death in an estimated 9.6 million patients annually [C1]. The most common cancers are of the lung, breast, colorectal area, prostate, skin (melanoma) and stomach. The treatment approach to cancer is multipronged, with chemotherapy, radiotherapy and surgery being the main arms of treatment. The immune system plays a major role in all aspects of cancer, including its origin, development, metastasis, therapy and prevention. Cancer cells and the immune system are in constant cross-talk wherein the cancer cells pass through three phases: i. Elimination, ii. Equilibrium, iii. Escape. In the elimination phase, the immune cells especially the innate immune cells are in constant surveillance eliminating cells that are altered from normal. The process of elimination makes the cancer cells to undergo immune-editing or sculpting with increase in number of cells that have decreased immunogenicity and become resistant to the immune surveillance process. This phase is the equilibrium phase. These cells which become resistant escape the immune system and develop into a full-blown cancer (Kim).

Pathogenic viruses such as SARS-CoV-2 are a constant threat to all human beings. In order to deal with the threat of viruses, it is important to have an immune system that prevents the invasion and proliferation of viruses in the living body at the forefront. However, it is known that the cytokine that controls one end of the immune system may cause a cytokine storm upon infection and falls into an uncontrollable state. Under such circumstances, substances capable of regulating the production of cytokines, the proliferation of immune cells, etc. have been searched for in various fields.

The outbreak of the ongoing COVID-19 pandemic started at the end of 2019, in the city of Wuhan, China. COVID-19 has been attributed to a novel type of coronavirus, termed by the WHO as the “novel coronavirus-2019” (SARSCoV-2). The genome sequence of SARSCoV-2 is similar to those of severe acute respiratory syndrome coronavirus (SARS-CoV) (approximately 79% homology), whose outbreak occurred in 2002 and 2003, and of Middle East respiratory syndrome coronavirus (MERS-CoV) (approximately 50% homology), whose outbreak occurred between 2012 and 2019. Coronavirus is member of the family Coronaviridae and subfamily Coronavirinae, which, based on genomic sequencing and phylogenetic relationships, consists of four genera: Alphacoronavirus, Betacoronavirus, Gammacoronavirus, and Deltacoronavirus. SARS-CoV-2 belongs to the Betacoronavirus genus (A1,2).

As of 3 May 2020, there have been 3,356,205 confirmed cases of COVID-19, including 238,730 deaths, reported to the WHO (A3). The incubation period of SARS-CoV-2 is 3-6 days, with the maximum being 14 days. The clinical signs and symptoms of COVID-19 include low to high fever, non-productive cough, myalgia, dyspnea, fatigue, standard or decreased leukocyte counts, and confirmed evidence of pneumonia on chest radiography. Less common symptoms of SARS-CoV-2 infection include headache, abdominal pain, dizziness, nausea, vomiting, and diarrhea. Regarding the therapeutic aspects, no specific therapy is currently available for COVID-19 (A4). Patients with mild signs and symptoms are treated with antibacterial drugs for pneumonia, including azithromycin, fluoroquinolones, and amoxicillin. Anti-viral agents such as viral methyltransferase inhibitor, nitazoxanide, the nucleotide prodrug GS-5734 Remdesivir, ribavirin in combination with lopinavir, interferon therapy, and convalescent plasma therapy are being tested for treating COVID-19. The case fatality rate (CFR) was reported to be between 2.3% (1,023 deaths among 44,672 confirmed cases) in China up to 15.80% in the UK (A4, 5) In particular, patients with co-morbid conditions are at higher risk of mortality from COVID-19 due to the conditions compromising their immune system (A4).

However, no composition is known that can obtain a simple and sufficient control effect by ingestion against pathogenic viruses such as SARS-CoV-2.

Here, we present the immune system implications of COVID-19 in the presence of co-morbidities and ways to enhance immunity, with a focus on nutritional supplements.

Conventional vaccine development to combat COVID-19 through different approaches are at various stages of progress. The complexity of COVID-19 such as (i) mutations of the virus leading to antigenic drift and an uncertainty on the duration for which the immunity induced by the vaccine may last, are considered major hurdles to a solution in the near future. In this background, we hereby suggest an alternate interim strategy based on biological response modifier glucans such as the Aureobasidium pullulans AFO-202 derived Beta Glucan which reportedly induce Trained immunity (TRIM) akin to Bacille Calmette-Guerin (BCG) vaccine by epigenetic modifications at central level in the Bone marrow. These beta glucans act as pathogen-associated molecular patterns (PAMP) activating the mucosal immunity by ligation of specific pathogen recognition receptors (PRR) such as Dectin-1 and activate both the adaptive & innate immunity by reaching distant lymphoid organs. Beta glucans have also been employed as immune adjuvants for vaccines such as the Influenza vaccine. Therefore, until a conventional vaccine is available, such orally consumable vaccine like biosimilars with track record of safety and potentials to produce long-lasting wide-spectrum immunity are worth in-depth research and upon validation can be considered for a clinical trial.

The COVID-19 pandemic is wreaking havoc on the lives of billions of people worldwide with unprecedented consequences and implications. COVID biology and pathology is very complex, thus posing a big challenge in clinical and drug management. Hence, researchers across the globe are creating strategies for developing drugs, antibodies, vaccines and other therapies to fight the deadly SARS-CoV-2 virus [B1]. Currently, over 124 vaccine candidates exist, with most concentrating on inducing neutralizing antibodies (nAbs) in the spike (S) protein on the virus's surface [B1,2]. Vaccine approaches conventionally use live attenuated viruses, inactivated virus protein, polysaccharide conjugated subunit vaccines, virus-like particles, nucleic acid (DNA and RNA) vaccines, viral vectors and recombinant proteins. The vaccine's ability to induce cellular immunity (other than B-cell produced antibodies) has been indicated as necessary for a rational vaccine design because nAb responses wane rapidly [B1]. Further, the coronavirus genome is highly prone to mutations that may lead to genetic drift and escape immune recognition-several variants that might cause drifts have already been identified [B3]. Undesired immunopotentiation in the form of eosinophilic infiltration or increased infectivity has hindered several vaccine candidates for COVID-19 and is currently a challenge in the vaccine biology [B3].

An ideal vaccine [B4] would fulfil all or most of the following criteria:
(i) offers wide-spectrum protection across various substrains and novel variants that are emerging or may emerge later;
(ii) possesses characteristics such as minimal undesired immunopotentiation;
(iii) suitable for stockpiling, for adult healthcare workers and for adults >60 years old or who have underlying diabetes or hypertension [B4];
(iv) produces long-lasting effective immunity in all vaccinated subjects across ages; and
(v) safe, stable and easily available and administrable.

Given the above criteria, we evaluated the suitability of beta glucans because they reportedly have several beneficial effects on human and animal health [B5].

Keywords: COVID-19, Vaccine, Beta Glucan, AFO-202 Beta Glucan, Trained Immunity

Severe acute respiratory syndrome coronavirus 2 (SARS-CoV 2), the novel virus behind the Coronavirus disease (COVID-19) pandemic is wreaking havoc around the world. Efforts are being continuously taken to understand the pathophysiological processes underlying the disease in order to mitigate the complications. While severe acute respiratory distress syndrome is the major cause of death, other organ failures such as cute kidney failure and acute cardiac injury also have been associated with the disease (a1).

Inflammatory response is highly increased in during COVID-19 infection and this process sets the stage for the organ failure to set in. Elevation of Th1 cytokine interferon (IFN)-gamma, inflammatory cytokines interleukin (IL)-1, IL-6 and IL-12, neutrophil chemokine IL-8, monocyte chemoattractant protein-1 (MCP-1), Th1 chemokine IFN-gamma-inducible protein-1 (a2) all leading to a cytokine storm (CS) termed as a macrophage activation syndrome (MAS) or secondary hemophagocytic lymphohistiocytosis (sHLH) which causes tissue damage (a3). Other immune dsyregulation related phenomena including complement activation also play a role in the virus causing organ failure. The host's innate and adaptive immunity must come into play encomapssing different aspects including the production of various proinflammatory cytokines, the activation of T cells, CD4 and CD8+ T cells for controlling the viral infection and downregulate the inflammation (a3).

Coagulopathy has been reported in patients with severe acute respiratory syndrome coronavirus 2 (SARS-CoV-2). While coagulopathy leading to venous thromboembolic events, end-organ failure secondary to a microangiopathy similar to disseminated intravascular coagulation and Stroke in COVID-19 have all been reported, all these sequalae will be expected in a patient with severe COVID-19. However, it is to be noted that even in in the absence of advanced COVID-19, large artery stroke has been reported (a4). Already, co-morbidities such as Diabetes, Hypertension (a5) and cardiovascular diseases (a6) have been associated with higher risk of complications and mortality due to COVID-19. Herein we report our perspectives on how people predisposed to coagulopathy and thrombogenic events could actually be the main target at high risk of complications due to COVID-19 and preventive measures such predisposed people can possibly undertake to be able to successfully defend their body from complications due to COVID-19.

### CITATION LIST

### Patent Literature

PTL 1: U.S. Pat. No. 6,956,120 B2
PTL 2: U.S. Pat. No. 10,307,479 B2

### PTL 3: CN 1697659 (A)

### PTL 4: US 20090053221 A1

PTL 5: U.S. Pat. No. 10,307,470 B2

### Non-Patent Literatures

NPL 1: Vetvicka V, Vetvickova J. Glucan supplementation enhances the immune response against an influenza challenge in mice. Ann Transl Med. 2015 February; 3(2):22. doi: 10.3978/j.issn.2305-5839.2015.01.08.
NPL 2: Jung K, Ha Y, Ha S K, Han D U, Kim D W, Moon W K, Chae C. Antiviral effect of Saccharomyces cerevisiae beta-glucan to swine influenza virus by increased production of interferon-gamma and nitric oxide. J Vet Med B Infect Dis Vet Public Health. 2004 March; 51(2):72-6. doi: 10.1111/j.1439-0450.2004.00732

### SUMMARY OF THE INVENTION

The COVID-19 pandemic has been causing varying severities of illness. Some are asymptomatic and some develop severe disease leading to mortality across ages. This contrast triggered us explore the causes, with the background that a vaccine for effective immunization or a drug to tackle COVID-19 is not too close to reality. We have discussed strategies to combat COVID-19 through immune enhancement, using simple measures including nutritional supplements.

An object of the present invention is to provide a composition that can be ingested conveniently, and has sufficient cytokine production control, immune cell control, blood coagulation inhibition etc., particularly a composition capable of treating or preventing a SARS-CoV-2 infection or a disease caused or exacerbated by the infection.

In order to solve the above object, the present inventors focused on the glucan produced by Aureobasidiums pullulans train FO-68 [(accession number) FERM BP-19327] and examined the utilization in detail.

As a result, the present inventors have found that orally ingesting glucan produced by FO-68 into humans has excellent effects such as immune cell regulation and cytokine production regulation. The present inventors have conducted further research and found that the glucan has a blood coagulation inhibitory effect, and completed the present invention.

Cancer is a deadly disease and is the second leading cause of death globally. Although continued efforts are put forth to identify effective treatments with fewer side effects, the incidence of cancer continues to rise, with reports suggesting that chronic microinflammation, which occurs in diseases such as diabetes, and weakened immune systems lead to cancer development, as well as genetic causes. In cancer patients, chemotherapy, radiotherapy and surgery are the mainstream approaches to treatment, but all of these therapies, including surgical intervention, lead to immune system weakness, which in turn has been proven to increase the metastatic spread.

Furthermore, chemotherapy-associated immunocompromise has been indicated as a wicket gate to the spread of cancer. Therefore, the prevention of cancer in the general population and its spread in those undergoing surgical or chemotherapeutic treatments can be possible only if immune system compromise and chronic microinflammation are well controlled. In this review, we present evidence of a biological response modifier (BRM), glucan. Glucan's beneficial effects of balancing metabolic parameters, such as blood glucose and lipid levels; increasing peripheral blood cell cytotoxicity against cancer; and alleviating chemotherapy side effects in animal models suggest it as a potential strategy to pave the way for a long-term prophylaxis in people with such specific conditions as immunocompromise or those who are genetically prone to cancer. The beta-glucans referred to have been reported with vaccine-adjuvant potential after necessary validations and could help conquer cancer through an immunoenhancement approach.

The present invention relates to the following:
1. A composition for inducing, enhancing and/or balancing an immune response, comprising a beta-glucan produced by Aureobasidium pullulans AFO-202 (FERM BP-19327).
2. The composition of item 1, which is used for treating and/or preventing a viral infection.
3. The composition of item 1, which is used for inhibiting blood coagulation.
4. The composition of any one of items 1 to 3, which is used for treating or preventing SARS-CoV-2 infection or a disease caused or exacerbated by the SARS-CoV-2.
5. The composition of item 1 or 2, which is used for treating and/or preventing cancer.
6. The composition of item 5, wherein cancer is renal carcinoma.
7. The composition of any one of items 1, 2, 5 and 6, which is used in alleviating chemotherapy side effects.
8. A vaccine adjuvant comprising a beta-glucan produced by Aureobasidium pullulans AFO-202 (FERM BP-19327).
9. The vaccine adjuvant of item 8, which is used for preventing and/or treating SARS-CoV-2 infection or a disease caused or exacerbated by the SARS-CoV-2.
10. The vaccine adjuvant of item 9, which is used for preventing and/or treating cancer or cancer in specific immunocompromised populations.

The present invention also relates to the following:
A1. A composition for regulating cytokine production, regulating immune cells, or inhibiting blood coagulation, comprising a glucan which is produced by Aureobasidium pullulans strain FO-68 [(accession number) FERM BP-19327].
A2. The composition according to claim A1, wherein the glucan produced by the strain FO-68 is β-1,3-1,6 glucan.
A3. The composition according to claim A1, wherein the regulating cytokine production has an antiviral cytokine-elevating effect.
A4. The composition according to claim A3, wherein the antiviral cytokine is type 1-IFN or IL-7.
A5. The composition according to claim A1, wherein the regulating cytokine production has an effect of maintenance or reduction of inflammatory cytokines.
A6. The composition according to claim A5, wherein the inflammatory cytokine is one or more cytokines selected from IL-1β, IL-6, IL-12 (p70+40), IFN-γ and TNF-α.
A7. The composition according to claim A1, wherein the regulating immune cells is an antiviral immune cell activation or proliferation effect.
A8. The composition according to claim A7 wherein the antiviral immune cell is one or more immune cells selected from NK cells, T cells selected from Th2, Treg, CD8 and CD4, Bcells, and dendritic cells.
A9. The composition of claim A1, wherein the blood coagulation is due to a viral infection.
A10. The composition according to claim A1, wherein the inhibition of blood coagulation is mediated by a maintenance or reduction effect of D-dimer or prothrombin.
A11. The composition according to claims A1-11, which is used for treating or preventing SARS-CoV-2 infection or a disease caused or exacerbated by the SARS-CoV-2 infection.
A12. The composition according to claim A11, wherein the disease caused by SARS-CoV-2 is thrombosis or thrombosis-mediated multiple organ failure.

The present invention also relates to the following:
[B1]. Adjuvant of enteric vaccine comprising beta-glucan derived from FERM BP-19327

The present invention also relates to the following:
[C1] A pharmaceutical composition for treating a cancer, comprising a beta-glucan produced by Aureobasidium pullulans AFO-202 (FERM BP-19327).
[C2] A pharmaceutical composition for inducing, enhancing and/or balancing an immune response in a subject, comprising a beta-glucan produced by Aureobasidium pullulans AFO-202 (FERM BP-19327).
[C3] A vaccine adjuvant for conquering cancer through immune-enhancement and/or for tackling cancer in specific immunocompromised populations, comprising a beta-glucan produced by Aureobasidium pullulans AFO-202 (FERM BP-19327).

### EFFECTS OF THE INVENTION

According to the present invention, a composition capable of regulating cytokine production, regulating immune cell and inhibiting blood coagulation can be provided. In particular, the regulation of the production of cytokines has the effect of increasing the production of beneficial antiviral cytokines such as type 1 IFN and IL-7 and decreasing the production of harmful inflammatory cytokines such as IL-6 and IL-12 etc. involved in cytokine storms.

Furthermore, the composition can enhance development and survival of mature T-cell, Fas production which prevents apoptotic cells thereby down-regulates inflammatory response, activating NK cells and CD8+cells, activating CD4+cells such as Th-1 cell, activating Treg cells for regulation and suppression of cytokine storm, and activating B cell thereby enhance producing virus-specific antibody (IgG, IgM, SIgA), via the above regulation of cytokines production or by an independent mechanism.

In addition, the present invention suppresses the decrease in blood fibrin during viral infection, thereby maintaining or decreasing D-dimer and thrombin, etc., and thus effectively inhibits blood coagulation due to the formation of thrombus and the like.

Therefore, the present invention can effectively treat or prevent viral infections, especially SARS-CoV-2 infections or the development or exacerbation of secondary diseases caused by such infections.

Further, the glucan contained in the composition of the present invention is derived from Aureobasidium pullulans, and its safety has been fully confirmed by long eating experience, thus it can be ingested safely and easily.

According to the present invention, vaccine adjuvant can be provided, which may be used for preventing and/or treating cancer, cancer in specific immunocompromised populations, SARS-CoV-2 infection, and/or a disease caused or exacerbated by the SARS-CoV-2.

### BRIEF DESCRIPTION OF DRAWINGS

FIG. 1 illustrates a graph showing an increase in Lymphocyte-to-CRP ratio (LCR) after 15 days.

FIG. 2 illustrates a graph showing a decrease in Neutrophil-to-Lymphocyte ratio (NLR) after 15 days.

FIG. 3 illustrates a graph showing an increase in ΔIgA after AFO-202 Beta-Glucan consumption.

FIG. 4 illustrates a graph showing an increase in ΔIgM.

FIG. 5 illustrates a graph showing an increase in CD11b in individuals who consumed AFO-202 Beta Glucan for 21 days (left) and a graph showing an increase in CD11b in individuals who consumed AFO-202 Beta Glucan for 35 days (right).

FIG. 6 illustrates a graph showing a decrease in C-reactive protein (CRP) after AFO-202 Beta-Glucan.

FIG. 7 provides a table showing an improvement in immune cell parameters in a patient with stage IV renal carcinoma.

FIG. 8 illustrates a graph showing an increase in mitochondrial ATPase.

FIG. 9 illustrates a graph showing an increase in mitochondrial DNA (ND1).

FIG. 10 illustrates a graph showing a decrease in IL-6.

FIG. 11 illustrates a graph showing a decrease in SARS-CoV2 (Novel Coronavirus) ACE2 receptor.

FIG. 12 illustrates graphs showing Corona virus receptor expression in HeLa cells.

FIG. 13 illustrates a graph showing an increase in CD69 antibody.

FIG. 14 illustrates a graph showing an increase in Anti-candida antibody.

FIG. 15 illustrates a graph showing an increase of ATP synthesis in cells.

FIG. 16 illustrates graphical representation of demarcating between the Upregulated immune enhancement factors and downregulated pro-inflammatory factors both being beneficial effects of AFO 202(F0-68) 1-3,1-6 beta glucan, a biological response modifier (BRM)

FIG. 17 illustrates the mechanisms and pathways that differ between oral and intra-dermal vaccines. Beta glucans interact with components involved in both the types of vaccines. A schematic illustration describes (i) stepwise, the mechanisms of orally administered vaccines starting from Peyers patches of the gut to induce mucosal immunity & parenteral vaccines starting from immune cells of the skin to induce systemic immunity, (ii) Strategic key advantages of the Beta glucans at five different stages and actions to play a role as vaccine and (iii) the Central trained immunity (TRIM) of bone marrow.

FIG. 18: Historical comparison of tumour size reduction with chemotherapy using cisplatin alone and when AFO-202 β-glucan is added as supportive agent to chemotherapy with cisplatin. Cisplatin chemotherapy decreases tumour volume by 12% (Ma et al. Aging and disease. 7. 254-66. 10.14336/AD.2016.0118.) When AFO-202 β-glucan is added to cisplatin chemotherapy, there is significant decrease in tumour size (49%) (Data from abstract presented by Mizobuchi S et al. Analysis of innate immune stimulating effect of Sofy β-glucan during chemotherapy The 108th Regular Academic Meeting of the Japanese Society of Surgery, 15 Mar. 2008; Japan).

FIG. 19: The line graph represents NK cell cytotoxicity in different age groups of healthy volunteers (Tada, Okumura: Gendai kagaku. Chemistry today; 11, 40 (1984)). The bar diagram represents NK cell cytotoxicity before (blue bars) and after (orange bars) AFO-202 beta glucan consumption (Data from abstract presented by Mio Miyamoto. 29th Annual Meeting of the Japanese Society of Venous and Enteral Nutrition (Feb. 27-28, 2014: Pacifico Yokohama), Japan). The percentage of NK cell cytotoxicity reported in the study by Okumura was, however, significantly lower than the values reported in the study in healthy volunteers analysing AFO-202 beta glucan consumption (Data from abstract presented by Mio Miyamoto), (p-value=0.001251).

FIG. 20: Historical comparison of percentage of NK cell cytotoxicity in cancer patients showing significant decrease in later stages of cancer (blue bars) (Konjevic Get al. Immunol Res 52, 139-156 (2012). https://doi.org/10.1007/s12026-012-8285-7) and the data of percentage of NK cell cytotoxicity before (grey bar) and after AFO-202-derived beta glucan consumption in elderly cancer patients (orange bar) (Data from abstract presented by Mio Miyamoto. 29th Annual Meeting of the Japanese Society of Venous and Enteral Nutrition (Feb. 27-28, 2014: Pacifico Yokohama), Japan.

FIG. 21: Four segments or time points during which a dent in the immune system is considered critical. At these times, the B-VACCINE approach could strategically could offer benefits as described above.

### DETAILED DESCRIPTION OF INVENTION

We herein focus our discussion on a specific beta glucan: a 1-3,1-6 beta glucan from a black yeast called Aureobasidium pullulans AF 202 strain (A41, 42). This 1-3,1-6 beta glucan is secreted extra-cellularly by Aureobasidium pullulans and is collected from the culture medium, without the need for additional purification (A43). Several studies have reported beta glucan to be a powerful immune stimulator that can activate macrophages and have positive immune actions on B-lymphocytes, natural killer cells, and suppressor T cells in the immune system (A44-46). These actions are not direct but rather due to beta glucan being a biological response modifier (BRM) to enhance immunity (A43).

This AF 202 beta glucan is also a biological response-modifier glucan (BRMG) whose biological response modifier (BRM) properties are significantly high (A43) due to it being an exopolysaccharide without additional purification steps, which may hamper this nature. As indicated by Vetvicka and Vetvickova in their conclusions (A37-39), since the AF 202 β-1,3-1,6-glucan is highly pure and active, it exerts significant immunological actions. This AF 202 β-1,3-1,6-glucan is recognized by the immune system as a PAMP equivalent and hence exerts immunological actions. This AF 202 β-1,3-1,6-glucan is a soluble beta glucan that contains both high and low molecular weight beta-glucan. High molecular beta-glucan (H-BG) component has been found to stimulate the proliferation of lymphocytes with stronger effects. On the other hand, low molecular beta-glucan (L-BG) component reduces the levels of the inflammatory biomarkers (majorly cytokines), and stimulation of cytokine and chemokine signaling pathways. In addition, L-BG effectively bound to dectin-1(β-glucan receptor), and has been shown to have antagonistic actions such as reactive oxygen production and cytokine synthesis from various immune cells such as macrophages, dendritic cells and endothelial cell etc. Since this beta-glucan contains both H-BG and L-BG it possesses ability to regulate whole of immune response for biological homeostasis (A43). Beta-1,3/1,6-glucan derived from yeast has been listed by the US-FDA under the generally recognized as safe (GRAS) category (A47). This AFO-202 Beta Glucan has been subjected to the following studies: genotoxicity test, single oral administration test, 28-day or 90-day repeated dose study, long-term oral administration test (1 year) and has been certified to be safe (A48). Also, this beta glucan has been available as a commercial food supplement for human consumption for the past two decades and is approved by the Japanese Ministry of Health(49).

Dectin-1 is a type II transmembrane receptor and the main beta glucan receptor involved in innate and adaptive immune responses to foreign antigens and pathogens; it is also the receptor for beta glucan as an immune function modulator (A43). Dectin-1 cooperates with pattern-recognition receptors (PRRs) and Toll-like receptors (TLRs) in the innate immune responses to beta glucan recognition. Ikewaki et al. reported that this AFO 202-derived beta glucan induces the production of IL-8 and sFas through cultured peripheral blood mononuclear cells (PBMCs) and U937 cells but does not stimulate the production of IL-1β, IL-6, IL-12 (p70+40), IFN-γ, or TNF-α and actually decreases IL-6 levels (A43). The enhancement of immune responses by AF 202 β1,3-1,6-glucan is associated with multiple signal transduction pathways involving several phosphoenzymes such protein kinase C (PKC), protein kinase A (PKA) inhibitor H-89 and protein tyrosine kinase (PTK) via intracellular mechanism(s). AFO 202-derived beta glucan was shown to induce DNA synthesis (cell proliferation) in PBMCs via Dectin-1, CD11a CD54 (intercellular adhesion molecule-1; ICAM-1), HLA-class II, TLR-2 and TLR-4 and to induce the production of sFas. AFO 202-derived beta glucan also stimulated U937 cells (a human monocyte-like cell line) to induce the production of sFas via Dectin-1, but not TLR-2 or TLR-4. The production of sFas by this beta glucan can prevent the onset of apoptosis, which is regulated by the Fas/FasL system, and can potentially downregulate inflammatory responses (A43). When explored, beta glucan in one-way human mixed lymphocyte reaction (MLR) assay systems could activate suppressor cells-in particular, regulatory T cells (Treg)—and also induce the production of suppressive cytokines (A43) which will be helpful in suppressing the cytokine storm observed in COVID-19. While the immunological actions of the AF 202 beta glucan are evident and will have potential use against COVID-19 infection by immunosuppressing pro-inflammatory cytokines, several studies have also reported that this beta glucan can enhance immunity by increasing the levels of cytotoxic cells such as NK cells and macrophages, which will be the actual line of defense against the viruses. NK cell activity was significantly increased by this beta glucan in patients with Leishmania amazonensis infection (A50). This beta glucan had regulatory or enhancing properties on poultry nonspecific cellular immunity in a study on Peking ducks (A51) and may enhance the immune response to avian influenza A H5 vaccine (A52). This AFO 202 beta glucan increased the NK cell and macrophage counts in cancer patients and elderly patients (A53). Glucan supplementation enhanced the immune response against an influenza challenge in mice (A54). In a study analyzing the efficacy of this AFO-202 beta glucan in protecting mice infected with a lethal titer of the A/Puerto Rico/8/34 (PR8; H1N1) strain of influenza virus, the survival rate was significantly increased by the beta glucan's administration after a sublethal infection of PR8 virus, and pre-treatment with beta glucan significantly repressed the replication of the PR8 virus (A55). Yeast (1,3)-(1,6)-beta-glucan also reduced the severity of upper respiratory tract infections in a double-blind, randomized, placebo-controlled study (A56).

The glucan contained in the composition of the present invention can be a glucan derived from Aureobasidium pullulans strain FO-68 (Also referred to herein as “strain AFO 202”), and preferably β-1,3-1,6 glucan derived from FO-68 (Also referred to herein simply as “glucan”, “AFO 202 glucan” or “AF 202 beta glucan”). “Aureobasidium pullulans strain FO-68” has been deposited at the Patent Biological Depository Center, National Institute of Advanced Industrial Science and Technology, under the deposit number FERMP-19327.

While the domestic deposition was made on Apr. 23, 2003, Aureobasidium pullulans strain FO-68 has then been transferred to international deposition at the International Patent Organism Depositary, National Institute of Technology and Evaluation (Room. 120, 2-5-8, Kazusa Kamatari, Kisarazu-shi, Chiba, 292-0818 Japan) on Apr. 21, 2021 with the accession number: FERM BP-19327.

Aureobasidium pullulans strain FO-68 is also called as Aureobasidium strain FERM P-18099.

### Scientific Properties of FO-68

This fungus produces high-molecular polysaccharide with high viscosity. This substance agglutinates easily with ethanol, making it possible to collect simply. This polysaccharide is of [beta] type, and is acidic polysaccharide having a main chain of 1,3 bond and branches from 3- and 6-positions. It contains carboxylic acids such as malic acid as organic acids and phosphoric acid. Moreover, it agglutinates easily with aluminum ions etc. This substance is also effective for the promotion of growth as a feed and the effluent treatment. It is effective as a food additive and functional food with immunity.

FO-68 forms blackish brown colonies on potato-dextrose-agar slant culturing for 7 days at 25 C. The fringe of colonies shows filamentous growth and becomes gradually light blackish brown. The cells are filamentous, and sometimes arthrospores, yeast-like budding conidiospores, oval yeast-like single cells, and, in some time, thick-walled spore cells are formed. The growth temperature is 25[deg.] C., and it decomposes hexoses such as glucose, fructose and galactose, sucrose, and starch. The medium becomes conspicuously viscous. Based on FO-68's mycological properties, it is a kind of Aureobasidium pullulans in the black fungus family of deuteromycetes.

Beta-glucans are naturally occurring polysaccharides obtained from obtained from different sources such as oats, barley, bacteria, yeast, algae, and mushrooms. Beta-glucans derived from different sources have differences in their structure which contribute to the different biological properties (A35). There has been nearly 7000 publications reporting the immune-modulating effects of β-glucans (A37). The immuno-modulating properties depend on the primary chemical structure of the β-glucans. β-glucans derived from fungi and yeast consisting of a (1,3)-β-linked backbone with small numbers of (1,6)-β-linked side chains, are essentially known for their immune-modulating effects (A36). Vetvicka and Vetvickova have published several studies (A37-39) comparing the immunological properties of different commercially available Beta Glucans in terms of effects on phagocytosis, IL-2 production, antibody secretion, Superoxide production, IFNγ production and inhibition of experimental cancer models, with their studies concluding that i. glucans in general have strong stimulating effects on most aspects of the immune system; ii. There are significant differences among tested glucans; iii. highly purified and highly active glucans have strong and pleotropic effects stimulating all facets of immunological reactions while poorly defined glucans have only medium (if any) biological effects. Beta glucans such as the pleuran from the mushroom Pleurotus ostreatus has been able to reduce the incidence of upper respiratory tract infection (URTI) symptoms and have increased the number of circulating NK cells (A40). Therefore, beta glucans will be important players in the immune system based fight against COVID-19.

### Mycological Features of the Isolated Fungus

A colony of FO-68 has a smooth surface at first and grows into a grayish white, mucous and glossy oil drop-like (fat-like), yeast-like material. The filamentous fungus body grows radially from the fringe thereof, leading to crinkled, filamentous and just dendritic growth. This filamentous fungus body grows well not only on the surface of medium, but also in the medium. In a short time, light dark brown specks appear here and there on the surface of colony, which become black specks gradually, and overall surface becomes dark black eventually. On this filamentous fungus body, a lot of light brown, elliptic or oval conidiospores are produced laterally. This conidiospore falls easily in pieces. While the surface of oil drop-like colony puts on the conidiospores here and there.

As a method for culturing FO-68 and a method for producing β-1,3-1,6 glucan using FO-68, known methods can be used, for example, see JP 2004-329077A.

In some embodiment, the present invention relates to a composition for inducing, enhancing and/or balancing an immune response, comprising a beta-glucan produced by Aureobasidium pullulans AFO-202 (FERM BP-19327). In another aspect, the present invention also relates to use of Aureobasidium pullulans AFO-202 (FERM BP-19327) for inducing, enhancing and/or balancing an immune response and particularly relates to a method of inducing, enhancing and/or balancing an immune response by administering Aureobasidium pullulans AFO-202 (FERM BP-19327) to a subject.

In the composition used in the present invention, a culture of FO-68 may be used as it is without purification, or glucan isolated from the culture or further purified as necessary may be used. In addition, for example, the culture product of the present invention was crushed into a concentrate, a paste, a spray-dried product, a freeze-dried product, a vacuum-dried product, a drum-dried product, a liquid product dispersed in a medium, a diluted product, and a dried product.

The composition of the present invention exerts its function when ingested by mammals including humans. The term “ingestion” as used herein is not limited to any administration route as long as it can enter the human body, and is realized by all known administration methods such as oral administration, tube administration, and enteral administration. Typically, oral ingestion and enteral ingestion via the digestive tract are preferable.

The dose of the present invention can be appropriately set in consideration of various factors such as administration route, age, body weight, and symptoms. The dose of the composition of the present invention is not particularly limited, but the amount of glucan is preferably 0.05 mg/kg/day or more, more preferably 0.5 mg/kg/day or more, particularly preferably 1.0 mg/kg/day. However, when ingested over a long period of time, the amount may be smaller than the preferable amount described above. In addition, the glucan used in the present invention has a sufficient dietary experience, and there is no problem in terms of safety. Therefore, an amount far exceeding the above amount (for example, 10 mg/kg/day) Or more).

The composition of the present invention can be used as a food or drink. The food or drink can be used, for example, as a food or drink having an antiviral action, particularly an anti-SARS-CoV-2 infection. The composition of the present invention, as a special-purpose food such as a food for specified health use and a nutritionally functional food, by administering to animals such as humans, treatment or prevention can be achieved against various infections or against the development or exacerbation of secondary diseases caused by the infection

By administering the composition of the present invention to animals such as humans as special-purpose foods such as foods for specified health use and foods with nutritive function, treatment or prevention for the occurrence or exacerbation of various infections or secondary diseases caused by infections.

By administering the composition of the preset invention to animals such as humans, treatment, prevention, and/or alleviation can be achieved against various diseases, disorders and conditions including blood coagulation, cancer, chemotherapy side effects, etc.

When the composition of the present invention is used as food or drink, the type of food or drink is not particularly limited. Further, the shape of the food or drink is not particularly limited, and may be any shape of food or drink that is usually used. For example, it may be in any form such as solid form (including powder and granule form), paste form, liquid form and suspension form, and is not limited to these forms.

When used as a pharmaceutical, a dosage form that can be orally administered is preferable because the composition of the present invention reaches the intestine. Examples of preferable dosage forms of the drug according to the present invention include tablets, coated tablets, capsules, granules, powders, solutions, syrups, troches and the like. These various preparations are prepared according to a conventional method by using glucan, which is the active ingredient, an excipient, a binder, a disintegrating agent, a lubricant, a coloring agent, a flavoring agent, a solubilizing agent, a suspending agent, a coating agent, etc. It can be formulated by admixing the auxiliaries usually used in the technical field of pharmaceutical formulation.

In some embodiment, the present invention can be used in combination with other food, drink, drugs and any other substances in order to enhance the efficacy of the present invention.

In some embodiment, an immune response is induced, enhanced and/or balanced by the present invention. Such a control in immune system has various applications such as treating and/or preventing a viral infection, SARS-CoV-2 infection, a disease caused or exacerbated by the SARS-CoV-2, and/or cancer, alleviating chemotherapy side effects, and inhibiting blood coagulation, etc.

The cytokine production control in the present invention has an effect of increasing the production of beneficial antiviral cytokines and decreasing the production of harmful inflammatory cytokines involved in cytokine storms.

The antiviral cytokine may be, but is not limited to, type I-IFN, IL-7 and the like. The inflammatory cytokine may be IL-1β, IL-6, IL-12 (p70+40), IFN-γ, TNF-α and the like. The production amount of these cytokines in a subject can be measured by a known method.

The regulation of immune cells in the present invention may be, without limitation, the activation or proliferation action of antiviral immune cells. The antiviral immune cell can be, without limitation, one or more immune cells selected from NK cells, T cells selected from Th2, Treg, CD8+, CD4+, B cells, and dendritic cells. B cells may comprise naive B cells, plasmablasts, and dendritic cells may comprise pDC, monocyte-derived DC, cDC, CD8+DC, CD11b+DC.

In one aspect of the present invention, the immune cells may be neutrophils, innate lymphocytes (ILC1, ILC2, ILC3), basophils, granulocytes, mast cells, hematopoietic stem cells, CLPs, mesenchymal stem cells. In one aspect of the invention, the regulation of immune cells may be suppression of Th1 activity or proliferation. The activity or proliferation of these cells can be measured by known methods.

The inhibition of blood coagulation in the present invention can be measured by a known method for the coagulability of blood collected from a subject. Without limitation, it is determined by measuring prothrombin time (sec), prothrombin time (% activity), thromboplastin time, thrombotest, fibrinogen amount, antithrombin III activity, thrombin/antithrombin complex amount, D-dimer, etc. Those skilled in the art can measure by known methods.

Coagulopathy has been indicated as a strong predictor of mortality and an indicator of disease severity of coronavirus disease (COVID-19). Herein we narrate our perspectives on possible pathophysiological mechanisms of the association between coagulopathy and COVID-19 wherein immune system related cytokine storm has been indicated as the major event causing dysregulation of the clotting mechanism and the second being direct endothelial injury due to viral invasion. Evaluation of D-Dimer and Prothrombin at admission will serve to predict prognosis and this has been recommended as an essential procedure. Ethnically pre-disposed populations to coagulopathy, the elderly and people with co-morbidities such as diabetes, hypertension and cardiovascular diseases form the vulnerable high-risk population. While thromboprophylaxis is recommended as per recent guidelines in all hospitalised patients of COVID-19, preventive strategies prior to hospitalisation have not yet been well studied. Supplementation by biological response modifiers especially in the vulnerable population is suggested to be of paramount importance in decreasing the development of severe COVID-19 and reducing mortality.

### Coagulopathy; a Precipitating Factor of Covid-19 and Combating Strategies

Coagulopathy—Key risk factor and predictor of severity of COVID-19; Perspectives on pathophysiological mechanisms and suggestive strategies for prior-hospitalisation prevention employing non-drug based biological response modifiers in the vulnerable population

### Coagulopathy and COVID-19—Pathological Mechanisms

In COVID 19, two separate pathologic processes have been found to play role in producing clinical manifestations of coagulopathy, I. Local direct vascular and endothelial injury by invasion of the virus producing microvascular clot formation and angiopathy, ii. Consequence of inflammation producing mononuclear and polymorphonuclear infiltration along with apoptosis of endothelial and mononuclear cells. Hypercoagulability with hyperfibrinogenemia causing large vessel thrombosis and major thromboembolic sequelae also are to be considered. Abnormally elevated d-dimer levels are the most common and critical feature observed in patients with coagulopathy related pre-disposition to COVID-19. elevated d-dimers has been associated with a poor prognosis. increased prothrombin times (PTs) and activated partial thromboplastin times (aPTT), lower platelet counts increased levels of lactate dehydrogenase (LDH) and ferritin are other associated findings reported in several studies (a7). Iba et al explain the four pathways by which coagulation related events and Thrombus formation in COVID-19. i. cytokine storm and pro-inflammatory cytokines such as interleukin (IL)-1β and IL-6 stimulating the expression of tissue factor on immune cells thereby initiating extrinsic coagulation cascade activation; ii. suppression of fibrinolytic system by the decreased activity of urokinase-type plasminogen activator and increased release of plasminogen activator inhibitor-1; iii activation of platelets by various proinflammatory cytokines and the damaged endothelium readily bind with the activated platelets; iv. direct endothelial damage induced by inflammation.

ACE2 receptors are expressed widely within endothelial cells which could explain their vulnerability to SARS-CoV-2 binding, membrane fusion and viral entry thereby leading to causing infection and direct vascular injury (a7). In a study it has been reported that the pre-existing increased plasmin activity occurring in hypertension, diabetes, and cardiovascular disease enhances the virulence and infectivity of the SARS-CoV-2 virus by cleaving of its spike proteins which in turn aggregates this coagulation related process (a8).

### Coagulopathy and COVID-19- Incidence & the Vulnerable Population

Since the thrombogenesis associated with COVID-19 is complex and very little understood, a potential distinct “COVID-19-induced coagulopathy pattern” has been postulated (a9). The first report of haemostasis disorders in patients with COVID-19 was by Guan et al. on 28 Feb. 2020 (a10). In this initial cohort of 1099 hospitalized patients with COVID-19, increased D-Dimer level above 0.5 mg/L was seen in 46.4% of the patients during initial presentation (a11). In 191 hospitalized patients with COVID-19, 81% of non-survivors had D-Dimer levels on admission greater than 1 mg/L (a12). A higher D-Dimer level was identified among 59.6% of severe infection compared to 43.2% non-severe COVID-19 patients. In fact, disseminated intravascular coagulation (DIC) has emerged as a strong predictor of mortality with 71.4% of non-survivors meeting criteria for DIC while only 0.6% of survivors met these criteria (a13).

Therefore, it is recommended that regular monitoring of D-Dimer, prothrombin and fibrinogen in COVID-19 is essential because significant increase in D-Dimer & prothrombin with decrease in fibrinogen has been observed at days 10-14 in non-survivors and elevated D-Dimer (above 1 microgram/ml) has been reported to be a strong independent risk factor in this vulnerable population (a10). Other reports on the incidence of coagulopathy in COVID-19 include one with 150 patients with Covid-19 among whom 25 patients (16.7%) experienced a pulmonary embolism and two patients had three thrombotic circuit occlusions (a14). Lupus anticoagulants were detected in 50 of 57 patients tested (87.7%) (a15). Oxley et al reported five patients with acute large vessel occlusion with ischemic stroke (a16). In the original cases reported from Wuhan China, stroke was seen in 5% of patients (a17). Another report indicated incidence of thrombotic complications of 16-49% in patients with COVID-19 admitted to intensive care (a18). In regard to deep-vein thrombosis (DVT), of the 143 patients hospitalized with COVID-19, 66 patients developed lower extremity DVT (a19).

Since most of the COVID-19 coagulopathy data is from Chinese patients owing to the first reporting of COVID-19 from China and because incidence of venous thromboembolism is approximately 3-4-fold lower in Chinese (a20), importance to the coagulopathy and thrombo-embolic events has been less in Chinese hospitals and use of thromboprophylaxis is also lesser. However, with the disease having affected the Caucasian individuals at a magnitude several times greater than China, it is essential to know the ethnicity related risk of thrombogenic events. Caucasians have higher thrombotic risk than the Chinese & other Asian populations and it is even higher in African-American patients (a21,22). Consistent to this, a study COVID-19 coagulopathy in Caucasian patients. Their findings showed that though the risk of coagulopathy was higher in Caucasians, since the patients included in that study were on LMWH thrombo-prophylaxis they rarely developed overt DIC and in cases where DIC developed it was during the later stages of the disease only. The study also reported that there was a novel pulmonary specific vasculopathy which we have termed pulmonary intravascular coagulopathy (PIC) associated with COVID-19 distinct from DIC (a23, 24).

Activation of innate immunity with older age and age-related coagulation cascade changes are also factors which have been reported to contribute to the vulnerability of elderly people to COVID-19 Coagulopathy (a25). Alveolar macrophages (AMs) increase during aging but their plasticity to convert between pro- and anti-inflammatory states is greatly reduced. This accelerates COVID-19 in its early stages in the elderly while in advanced stages causes excessive lung damage. Decline in neutrophil activity during aging makes these cells lose their ability to migrate to sites of infection and kill infected cells. Production and diversity of mucins and protective glycoproteins contributing to mucosal barriers also change in aging (a26). Immunosenescence of the adaptive immune system in the aged also contribute to progression to severe COVID-19 in the aged. A decline in the production of fresh naive T cells, a less expansive T cell receptor (TCR) repertoire, T cell metabolic dysfunction, and weaker activation of T cells also contribute to immune vulnerability of the aged to COVID-19. When the link between immune system and coagulopathy in the aged which makes them, a vulnerable population was explored, it is identified that one in two fatal cases of COVID-19 experience a cytokine storm among whom 82% are over the age of 60.

Inflammaging is a major driver for this increased cytokine storm occurring in aged individuals, exacerbated by obesity, poor diets and oral health, microbial dysbiosis, and sedentary lifestyles. Age related correlation of higher basal circulating levels of pro-inflammatory cytokines including IL-6, TNF-α, IL-1α and CRP is a reported phenomenon. NLRP3 a major protein component of the inflammasome is increased with aging. NLRP3 are at risk of hyperactivation by SARS-CoV-2 antigens which is further aggravated by decreased activity of sirtuin 2 (SIRT2) in the aged as sirtuin 2 (SIRT2) directly controls NLRP3 (a25). This cytokine storm disrupts the feedback control mechanisms of Thrombin generation by antithrombin III, tissue factor pathway inhibitor, and the protein C system which predisposes to the development of microthrombosis, disseminated intravascular coagulation (a27).

Coagulopathy seems to be the central factor predicting progression of COVID 19. It also explains why children rarely get a severe illness due to COVID-19 as thrombotic complications in the paediatric age group are rare in the absence of an underlying cancer or a central venous access device. While pregnant women should be expected to have high vulnerability to coagulopathy actually have been found to have only milder illness because of immune suppression in them during the gestational period to avoid foetal rejection and thereby the immunothrombosis does not come into play (a28).

Vulnerability of patients with cardiovascular risk factors such as obesity, hypertension, and diabetes to disease severity of COVID-19 is well established (a25). Imbalance between coagulation and fibrinolysis with increased levels of clotting factors and relative inhibition of the fibrinolytic system, endothelial dysfunction, enhanced platelet aggregation and activation which are complications of Diabetes favour the development of a hypercoagulable pro-thrombotic state thus explaining the vulnerability quotient of Diabetes patients to COVID-19 in terms of disease severity (a29).

In regard to hypertension, other cardiovascular diseases, in addition to alteration in vascular & thrombogenic factors, pulmonary and peripheral endothelial injury due to direct viral attack and the cytokine storm have been indicated as the inducer of hypercoagulation in these patients (a30).

### Preventive and Therapeutic Aspects of Coagulopathy in COVID-19

Having identified the various vulnerable populations to coagulopathy associated severity of COVID-19, we turn toward the possible therapeutic solutions and preventive strategies. Recent guidelines recommend thromboprophylaxis for all hospitalized COVID-19 patients or full therapeutic-intensity anticoagulation (a26). Antiplatelet drugs, Thrombolysis, immunomodulatory agents and anti-complement drugs are suggested approaches. For anticoagulation, the drug of choice is low molecular weight heparin and in patients who may have severe renal impairment or extremely high risk of bleeding, unfractionated heparin is recommended (a28). Preventive aspects deal about treating the co-morbidities and ends with a maximum of thromboprophylaxis but all these are suggested after a patient is hospitalised.

We explored if any other preventive strategy is available. Supplementation with biological response modifiers may be a solution in the vulnerable population. Beta Glucans are potent biological response modifiers. Soluble Beta 1,3 Glucans have been found to decrease septic complications and improve survival by acting on cytokine production and regulating coagulation activation (a32). Radiation exposure and/or diabetes induced oxidative stress which caused disturbances in the measured clotting parameters by enhancing platelet aggregation (PA) and increased thrombin levels were reversed by a yeast beta glucan (a33). A biological response modifier glucan (BRMG) from a black yeast Aureobasidium pullulans has been reported to be a potent immune-modulator acting through its receptor Dectin-1 which cooperates with pattern recognition receptors (PRRs) and Toll-like receptors (TLRs) in the innate immune response. This BRMG reduces the levels of IL-1β, IL-2, IL-4, IL-6, IL-12, TNF-α, IFN-γ and sFasL while increasing IL8 and sFAS. This would directly relate with the key-action needed in COVID-19 therapeutic response which is attenuation of the cytokine storm caused by pro-inflammatory cytokines like IL-6. This BRMG has also been reported to NK cell activity and macrophage activity thereby contributing to anti-viral response (a34). This BRMG which helps in maintenance of blood glucose and lipid levels as well that could serve to help to treat co-morbidities associated risk of COVID-19 severity (a35,36) is thus suggested as a possible preventive strategy to combat coagulopathy induced risk of COVID-19 severity in the vulnerable populations described above.

The composition of the present invention can be used for treating or preventing a viral infection or a secondary disease caused by a viral infection. The virus is preferably, but not limited to, a virus belonging to the coronavirus family, and SARS-CoV-2, SARS-CoV, MERS-CoV, human coronavirus HKU1, and human coronavirus OC43 belonging to the genus beta-coronavirus are preferable.

It may be beneficial for SARS-CoV-2 infection or secondary diseases caused by SARS-CoV-2 infection, especially thrombosis.

The subject to be ingested may be in any state, but from the viewpoint of reducing the risk of death of SARS-CoV-2, it is preferably to patients suffering from a basic disease such as chronic kidney disease, diabetes, heart disease and the like.

### COVID-19 and the Immune System

A characteristic feature of COVID-19 infection is a pro-inflammatory status characterized by high levels of different cytokines, including interleukin (IL)-1β, IL-1Rα, IL-2, IL-10, fibroblast growth factor (FGF), granulocyte-macrophage colony stimulating factor (GM-CSF), granulocyte-colony stimulating factor (G-CSF), interferon-γ-inducible protein (IP10), monocyte chemoattractant protein (MCP1), macrophage inflammatory protein 1 alpha (MIP1A), platelet-derived growth factor (PDGF), tumor necrosis factor (TNFα), and vascular endothelial growth factor (VEGF). Furthermore, critically ill patients requiring admission to the intensive care unit (ICU) were found to have markedly high concentrations of IL-2, IL-10, G-CSF, IP10, MCP1, MIP1A, TNFα, and IL-6. Importantly, increased levels of IL-6 are also correlated with increased mortality. In severe COVID-19, a reduction of natural killer cells-CD4+ and CD8+ T lymphocytes-and IFN-γ expression in CD4+ cells have been observed, along with hampered adaptive immune systems due to cytokine release syndrome, which can be attributed to the inverse correlation of levels of IL-6, IL-10 and TNFα with lymphocyte count (A6-8). In another report, lymphopenia with drastically reduced numbers of CD4+ T cells, CD8+ T cells, B cells and natural killer (NK) cells was reported to be a common feature in patients with severe COVID-19, which was not observed in milder cases. In addition, the numbers of CD4+ T cells, CD8+ T cells, B cells, and NK cells are normalized in patients who have recovered or are convalescent. The exhaustion markers, such as NKG2A, on cytotoxic lymphocytes, including NK cells and CD8+ T cells, were increased in those with severe disease and returned to normal levels after recovery from COVID-19 (A9). Increased neutrophil-to-lymphocyte ratio (NLR) and low lymphocyte-to-C-reactive protein ratio (LCR), reflecting an enhanced inflammatory process, have been reported to suggest a poor prognosis in patients with severe COVID-19 (A10).

Thus, in summary, in the case of the inflammatory pathway leading to cytokine storm, pro-inflammatory factors such as IL-6, IL-8, IL-1β, and GM-CSF and chemokines such as CCL2, CCL-5, IP-10, and CCL3, together with reactive oxygen species have been attributed to cause Acute Respiratory Distress Syndrome (ARDS) leading to pulmonary fibrosis and death. In COVID-19, high levels of serum pro-inflammatory cytokines (IFN-γ, IL-1, IL-6, IL-12, and TGFβ) and chemokines (CCL2, CXCL10, CXCL9, and IL-8) have been reported to be detected in cases of severe disease compared to patients with uncomplicated SARS (A11). While suppression of this pro-inflammatory cytokine storm is considered essential to combat COVID-19, some cytokines such as Type-I interferon and IL-7 have been found to be beneficial. Several studies are being conducted to study the effectiveness of IFN-α and IFN-β as drugs against SARS-CoV-2. Since lymphopenia and lymphocyte exhaustion are hallmarks of COVID-19, IL-7—the major cytokine promoting lymphocyte expansion and possibly reversal of T cell exhaustion is considered to be useful in restoring immune system homeostasis. Intriguingly, in sera of patients with mild/moderate or severe forms of COVID-19, IL-2 and IL-7, the cytokines responsible for expansion and differentiation of various T cell subsets are found in increased levels most likely representing attempts by the immune system to reverse lymphopenia and T cell exhaustion (A12).

### Immune System Implications of Cardiovascular Diseases and COVID-19

The CFR of 2.3% in China was found to be elevated to 6.0% for patients with hypertension, 7.3% for patients with diabetes, and 10.5% for patience with CVD (A13). Multiple studies have reported that patients with underlying cardiovascular comorbidities are at higher risk of severe COVID-19 infection that requires ICU care and of having complications like acute respiratory distress syndrome (ARDS), which may result in death. The mechanism has been attributed to reduced or impaired cardiovascular functional reserves in such CVD patients, which is worsened by the myocardial infarction precipitated by COVID-19, leading to increase myocardial demand, the worsening of ischemia and necrosis or, an increase in metabolic demand, which leads to heart failure and death. COVID-19 infection indirectly causes cardiac injury due to an overwhelming immune inflammatory response and cytokine storm. Other proposed mechanisms include SARS-CoV-2 viral invasion and direct damage of cardiomyocytes, as well as myocardial injury arising from severe hypoxia due to acute respiratory damage and also from another important process concerning angiotensin-converting enzyme 2 (ACE2) which is expressed in the heart and which SARS-CoV-2 uses as a receptor for entry into the cell (A14). Among the cytokines that are elevated in the cytokine storm in COVID-19, IL-6 is important because it is the most strongly associated cytokine with coronary heart disease (CHD) (A15). Interleukin-1b (IL-1b), tumor necrosis factor (TNF), and IL-17 have also been reported to be effective targets that can reduce cardiovascular progression (A16).

### Immune System Implications of Diabetes and COVID-19

According to Yang et al. (A17), among those studied who died due to COVID-19, 22% had cerebrovascular diseases and 22% had diabetes. A study of 1,099 patients with confirmed COVID-19 showed that among 173 who had severe disease, 23.7% had comorbid hypertension, 16.2% also had diabetes mellitus, 5.8% also had coronary heart disease, and 2.3% also had cerebrovascular disease. In another study among 140 patients who were admitted to the hospital for COVID-19, 30% had hypertension, and 12% had diabetes (A18). The mechanisms proposed for increased susceptibility of patients with diabetes to COVID-19 include “1) higher affinity cellular binding and efficient virus entry, 2) decreased viral clearance, 3) diminished T cell function, 4) increased susceptibility to hyperinflammation and cytokine storm syndrome, and 5) presence of CVD” (A19). When the cytokine profile in diabetes was analyzed in relation to COVID-19, the focus was again on IL-6, which was reported to play a more deleterious role in COVID-19 infection (A19).

### Immune System Implications of Chronic Kidney Disease and COVID-19

Chronic kidney disease, particularly patients with end-stage renal disease (ESRD) who are dependent on dialysis, are also in the high-risk category of acquiring severe disease and mortality due to COVID-19. When the underlying immune profile was analyzed, it was observed that cytokines such as interleukin-1 beta (IL-1 beta), tumor necrosis factor-alpha (TNF-alpha), and IL-6 induce an inflammatory state, playing a significant role in dialysis-related morbidity (A20), again pointing to IL-6. In another report, between 30% and 50% of hemodialysis patients had elevated serum levels of inflammatory markers such as C-reactive protein and IL-6 (A21). Furthermore, CKD has been associated with increases in immune senescence (A22) and inflammation biomarkers (A23).

### Immune System Implications of Cancer and other Forms of Immunosuppression in COVID-19

Among 1,590 cases with confirmed COVID-19, Liang et al. found that 18 patients had a history of cancer. They concluded that patients with cancer had a higher risk of COVID-19 and poorer prognoses than those without cancer (A24). While over-whelming inflammation and cytokine-associated lung injury are associated with the severity of COVID-19 in cancer patients according to Liang et al. (A25), Xia et al. (A26) pointed out that the blunted immune status, characterized by overexpressed immunosuppressive cytokines, suppressed induction of proinflammatory danger signals, impaired dendritic cell maturation, and enhanced functional immunosuppressive leukocyte populations, could be the actual underlying factors exacerbating the severity of COVID-19 in cancer patients (A25). Importantly, immunocompromised patients often present atypical presentations of viral diseases such as COVID due to the altered nature of the immune system (A26). Among 10 kidney transplant recipients on immunosuppression who tested positive for SARS-CoV-2 by PCR, nine were admitted as in-patients, three patients (30%) died, and five (50%) developed acute kidney injury (A27). A review of 89 studies on immune-suppressing or -stimulating drugs showed no conclusive evidence on the benefits of cytotoxic chemotherapy against COVID-19 infection (yet such benefits were observed during in vitro studies) or against the use of non-steroidal anti-inflammatory drugs (NSAIDs) and TNFα blockades. While clear evidence existed of an association between IL-6 peak levels and the severity of pulmonary complications, no evidence showed a beneficial impact of IL-6 inhibitors on modulating COVID-19 (A28). Thus, immunosuppression has been reported to help with COVID-19, in lieu of the cytokine storm, and a kidney-transplanted patient infected with SARS CoV2 manifested only mild disease (A29), thus adding more prominence to the fact that hyper-inflammation is the mechanism underlying COVID-19 progression. This is a major research area open for future perspectives.

### Immune Strategies for Combatting COVID-19

Having outlined the immune system characteristics in COVID-19 especially with co-morbidities, let us focus on the strategies for equipping the immune system to fight COVID-19. Innate immune response is highly critical for fighting viral infections and is decidedly dependent on interferon (IFN) type I responses, whose downstream cascade controls viral infection along with the induction of effective adaptive immune response. Innate immune cells recognize a virus's invasion through pathogen-associated molecular patterns (PAMPs), in the form of viral genomic RNA or the intermediates during viral replication, including dsRNA (A30). This recognition event leads to the downstream signaling cascade being activated, which culminates in the expression of type I IFN and other pro-inflammatory cytokines. This initial response comprises the first line defense against viral infection at the entry site. For SARS-CoV and MERS-CoV, the response to viral infection by type I IFN is actually suppressed, which is closely associated with the disease's severity. SARS-CoV-2 also utilizes similar strategies of dampening type I IFN response. Furthermore, dysregulated type I IFN and inflammatory monocyte-macrophage influxes are the main cause of lethal pneumonia. Hence, the proposed immune combat strategies for COVID-19 involve suppressing of the cytokine storm by employing antagonists of some key pro-inflammatory cytokines, increasing the beneficial cytokines such as IL7, Type I IFN, along with treatment using anti-viral agents (A30). While innate immune system-based strategies are key to therapeutics, an adaptive immune system holds the key to vaccine development (A30). We postulate that a simpler and more effective approach will be nutritional intervention.

### Preventive and Therapeutic Nutritional Interventions for COVID-19

Supplementation of Vitamins A and D enhances immune response to influenza virus vaccination (A31). Although Vitamin C is widely believed to help prevent viral infections, especially the common cold, a literature review of 640 studies failed to identify any conclusive evidence for Vitamin C prophylaxis in preventing the common cold (A31, 32). Supplementation with micronutrients has mixed results. One RCT on 725 institutionalized elderly patients showed that low-dose supplementation of zinc together with selenium enhanced the humoral response after vaccination, in comparison to the control group (A33), while in another RCT, neither daily multivitamin-mineral supplementation nor vitamin E (200 mg/day) showed a favorable effect on the incidence and severity of acute respiratory tract infections in well-nourished non-institutionalized elderly participants (A34).

Nutraceuticals provide relief to people infected with encapsulated RNA viruses, such as influenza and coronavirus, by boosting their immune responses.

### AFO 202 Beta Glucan and Relevance to COVID-19 Patients with Co-Morbidities

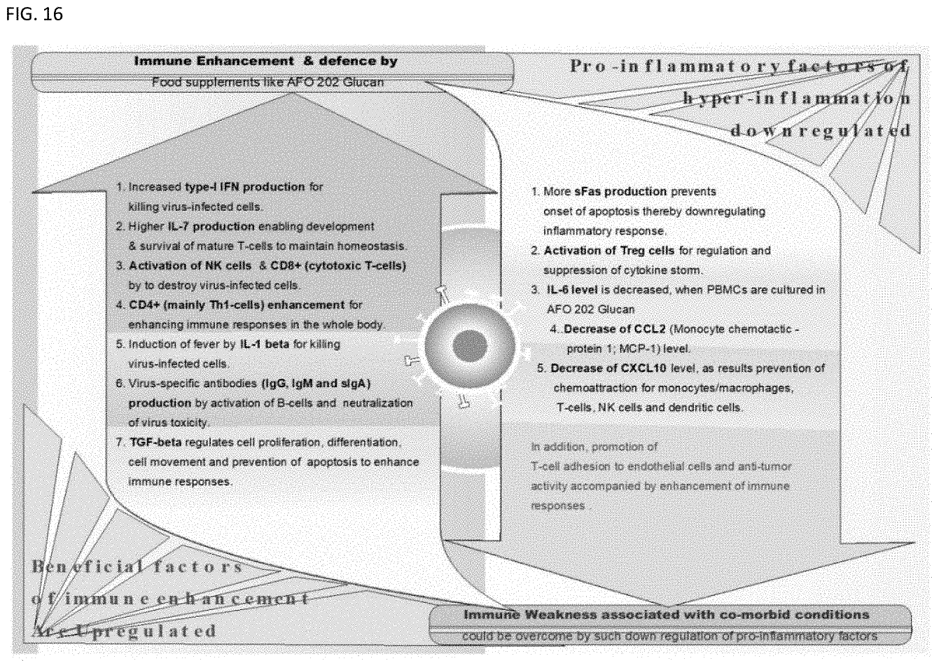

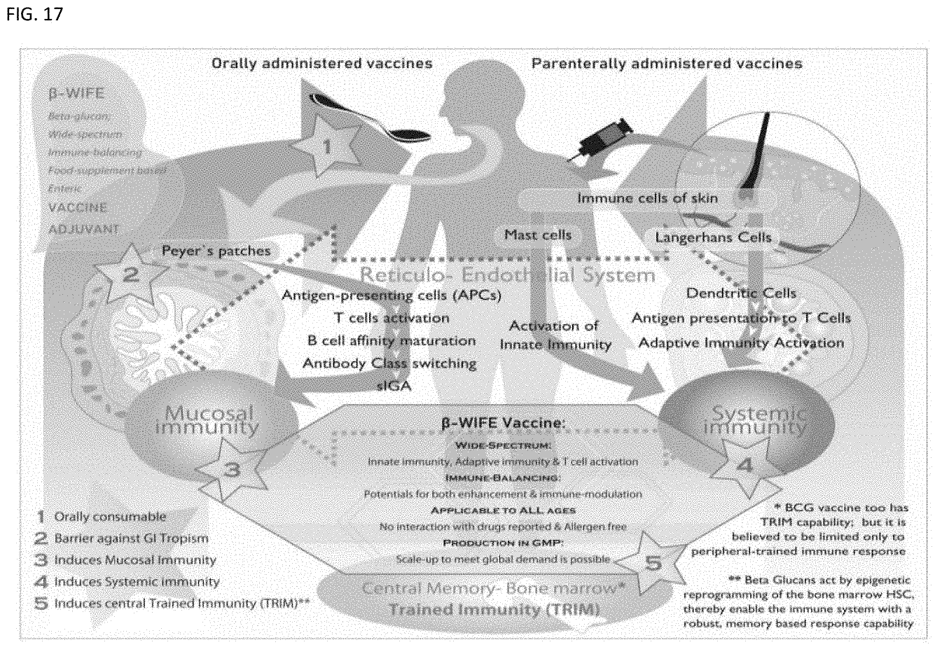

While AFO 202 beta glucan supplementation can be a potential strategy to fight COVID-19 infection due to its immune-enhancing activity in terms of IFN-γ-increasing capability (A35), whose suppression is characteristic of SARS-COv2 infection (A30), its consumption should be emphasized for people with comorbidities. IL-6 is the most commonly elevated cytokine in cytokine storms from conditions involving chronic micro-inflammation, such as CVD, diabetes and CKD (A12, 13, 17, 18). This AFO 202 beta glucan decreases IL-6 levels (A43). The increase in sFAS, which helps in regulating the immune response by immune suppression, will be highly valuable in regulating the cytokine storms and hyper-inflammation associated with COVID-19 (A43). In regard to the pro-inflammatory and beneficial cytokines listed in the introduction section of the manuscript (A11,12), AFO 202 beta glucan through IL8 causes activation, immigration and chemotaxis of neutrophils for killing virus-infected cells. This beta glucan also causes decrease of CCL2 (Monocyte chemotactic protein 1; MCP-1) and decrease of CXCL10 levels, as result of which there will be prevention of chemoattraction for monocytes/macrophages, T-cells, NK cells and dendritic cells thereby suppressing immune response. In addition, promotion of T-cell adhesion to endothelial cells and anti-tumor activity accompanied by enhancement of immune responses occur. Increase of type-I IFN production by AFO 202 beta glucan helps in killing virus-infected cells (A43). Further, increase of IL-7 production leads to development and survival of mature T-cells to maintain homeostasis. Activation of CD8+ (cytotoxic T-cells) helps in anti-viral immunity while activation of CD4+ (mainly Th1-cells) and Treg cells helps in regulatory immune response and suppression of cytokine storm with severe inflammation. Activation of B-cells results in production of virus-specific antibodies (IgG, IgM and sIgA) for neutralization of virus toxicity (A43). FIG. 16 illustrates the effects of the AFO 202 Beta Glucan on suppression of pro-inflammatory factors and enhancement of beneficial factors in COVID-19 in a nutshell.

The regulatory immune profile enhanced by AFO-202 beta glucan (A35) will assist with immune modulation in patients with cancer. For kidney transplant recipients and patients with immune suppression, the NK cell- and macrophage-enhancing activity will come to play in antiviral immunity (A43) thereby helping them to fight COVID-19.

Another interesting aspect is that gut microbiota can influence the generation of innate memory and the functional reprogramming of bone marrow progenitors, which can help to protect against infections (A57). Their dysbiosis also leads to variety of immune-mediated inflammatory disorders (A57). Beta glucan's beneficial effects in reducing diseases like CVD have also been attributed to their action influencing gut microbiota (A58), and the immune-modulation of beta glucans by acting on gut microbiota can help to alleviate an inflammatory immune profile (A59), thereby leading to beneficial effects. These advantages mean that the beta glucan should more importance in fighting COVID-19, specifically in the presence of chronic inflammation-associated comorbidities. Thus, for those at high risk who suffer from a wide range of comorbidities, consumption of this food supplement-whose safety has been proven by consumption over two decades (A43, 47, 48)-will be a prospective preventive option and even as a supportive choice during therapeutics in the fight against this deadly COVID-19 pandemic.

### Limitations and Hurdles in Considering AFO 202 Beta Glucan Type of Nutritional Supplements in COVID-19

The variation is susceptibility of individuals to COVID-19 has been attributed to some inherent factors such as genetic variation in terms of human leukocyte antigen (HLA) polymorphisms (A60), mutations in ACE-2 gene (A61), age, general health and nutrition (A62). Acquired variations such as cross immunity provided by vaccines such as BCG vaccine (A63) and Japanese encephalitis (JE) vaccine (A64) against COVID-19 are other aspects. While co-morbidities influence the clinical course of COVID-19, since nutritional supplements such as AFO 202 beta glucan have not been actually tried in patients with COVID-19, it is prudent to emphasize that the actual clinical practicality of employing these nutritional supplements including AFO 202 beta glucan as a routine measure for preventive and therapeutic support in COVID-19 will be recognized only if it is applied in different clinical presentations of COVID-19 especially in people with different variants that influence disease susceptibility. Further, once the disease becomes established in terms of respiratory illness, multi-organ dysfunction (A65), macrovascular stroke (A66) etc., though nutritional support by supplements including AFO-202 beta glucan is recommended enabling the immune system to be back on track against the infection, how far it is viable in reality needs validation.

In some embodiment, the present invention relates to a vaccine adjuvant comprising a beta-glucan produced by Aureobasidium pullulans AFO-202 (FERM BP-19327), which can be used for preventing and/or treating SARS-CoV-2 infection or a disease caused or exacerbated by the SARS-CoV-2, and/or for preventing and/or treating cancer or cancer in specific immunocompromised populations.

### Beta Glucans and Immunity

Trained immunity (TRIM) induction is a promising defence strategy against COVID-19 [B6]. The widely known Bacille Calmette-Guerin (BCG) vaccine induces TRIM, protects against severe forms of Mycobacterium tuberculosis (TB)—with limited effect against pulmonary tuberculosis-and confers non-specific protective effects against unrelated infections and mortality [B6]. BCG's non-specific protection influence is T- cells and B-cells independent and is mediated by the functional and epigenetic reprogramming of innate immune cells such as monocytes, macrophages, and NK (natural killer) cells, which and this protection is called TRIM.

β-glucans are a heterogenous group of polysaccharides abundant in the cell walls of yeast, bacteria and fungi that reportedly induce TRIM, but their induction mechanisms differ from BCG. Beta Glucans induce epigenetic reprogramming in innate immune cells, leading to cellular activation, augmented cytokine production and changes in metabolic function that shift cellular metabolism from oxidative phosphorylation to glucose fermentation mediated via the Akt/mTOR (mammalian target of rapamycin)/HIF1α (hypoxia-inducible factor 1α) pathway [B7], which and this metabolic shift is a key factor for effectively inducing TRIM. The epigenetic alterations histone methylation and acetylation lead to the positive regulation of gene expression. When such epigenetically “trained” cells have contact with heterologous secondary stimuli, they are programmed to produce a more robust immune response [B4,7]. The cells are reportedly not peripherally trained, but beta glucans may impact the bone marrow (BM) and lead to a lasting TRIM phenotype. Administering intraperitoneal beta glucan specifically expanded Lin-Sca1+cKit+(LSKs) and Multipotent Myeloid Progenitor 3 (MPP3) hematopoietic stem cells (HSC) in the BM, and such trained HSCs generate a “central” memory [B7]. Epigenetic modifications driven by β-glucan are rapidly activated by secondary infections or stimuli, like viruses, and therefore serve as a potent strategy for vaccines against COVID-19 [B4]. Beta glucans act as pathogen-associated molecular patterns (PAMP) because they are present in the cell wall of some pathogenic yeasts and bacteria, thereby causing microorganism recognition and clearance by the human immune system. Upon reaching the intestine, beta glucans are internalized by intestinal epithelial and/or M cells, which are then presented to immune cells within the Peyer's patches. Beta glucan particles can also reach distant lymphoid organs via blood or lymph. In the peyer patches, Beta glucan particles are recognized by the ligation of specific pathogen recognition receptors (PRR), such as toll-like (TLR) and C-type lectin-like receptors. Among C-type lectin-like receptors, Dectin-1 is the most-studied receptor that binds beta glucan from various sources. Dectin-1 is expressed on the surface of monocytes, macrophages, neutrophils, Dendritic cells and T lymphocytes, which are all activated by beta glucan binding. This binding leads to a number of cellular responses via the modulation of inflammasome and transcription factor activation, which leads to the production of cytokines, chemokines and reactive oxygen species (ROS). Beta glucans stimulate NK cell cytotoxic activity, as part of the innate immune response by binding directly to the NKp30 activating receptor [B8, 9]. Beta glucans' innate immunity targets are monocytes, macrophages, dendritic cells and NK cells. Beta glucans activate the antimicrobial activity of mononuclear cells and neutrophils as well [B8]. Regarding T cells, beta glucans help CD4+ T cell immunomodulation infiltrate tumours, thus inhibiting tumor growth [B10]. Orally administered beta glucans have reached spleen and lymph nodes, activating DCs and thus expanding and activating antigen-specific CD4 and CD8 T cells and IFN-γ production, which significantly reduced tumour burdens [B11]. Beta glucans also induce B lymphocytes to produce antibodies. Short-term supplementation with beta glucan improved the levels of salivary immunoglobulins (sIgM, sIgG and sIgA) [B12]. Orally administered beta glucan significantly stabilized IgG1 levels, thus maintaining anti-infectious immunity. Thus, all aspects of the immune system are activated and modulated by beta glucans, making them worth considering as an ideal vaccine that produces long-lasting effective immunity, is broadly protective against all variants of an organism, is effective in all vaccinated subjects across ages and is stable and easily administrable [B13]. Long-lasting immunity is currently a big challenge in COVID-affected patients [B14]. Beta glucans have experimentally proven to produce long-lasting trained immunity against a wide range of pathogens [B15]. Furthermore, beta glucans are safe for consumption by all ages of people, and they fall under the FDA's generally recognized as safe (GRAS) category [B16]. Beta glucans are stable and can be consumed continuously as a food supplement [B5]. Many types of beta glucans exist, but yeast- and mushroom-derived beta glucans have had profound immune-system effects compared to other types of beta glucans. Oral beta glucans have been thoroughly described as a prophylactic supplement to boost immune responses and to abrogate COVID-19 symptoms via its TRIM actions [B5]. Though SARS-Cov-2 is predominantly considered a virus that affects the respiratory system, the viral host receptor ACE2 appears in the cytoplasm of gastrointestinal epithelial cells, with the viral nucleocapsid protein appearing in the cytoplasm of rectal, duodenal and gastric epithelial cells-suggesting that the intestine could be relevant in the pathogenesis of COVID-19 and as a possible route of infection [B17]. Beta glucans with immune effects that start with the intestine could therefore be an advantageous supplementation strategy for COVID-19. Gut-dysbiosis is also a key element in determining infection-related diseases. Beta glucans can also modulate the gut bacteria, further improving immune response [B18]. Beta glucan supplements have decreased the incidence of upper respiratory tract infections in several studies, including in randomized control trials [B19-22]. A beta glucan extract from edible shiitake mushroom Lentinus edodes has recently produced differential in-vitro immunomodulatory and pulmonary cytoprotective effects and is indicated for COVID-19 immunotherapy. The study compared two kinds of Lentinan extracts that differentially reduced cytokine-induced NF-_{κ}B activation in human alveolar epithelial A549 cells and that attenuated pro-inflammatory cytokine production (TNF-α, IL-8, IL-2, IL-6, IL-22) as well as TGF-β and IL-10. The study suggested that beta glucans delivered as a tailored cocktail fit future nutraceutical-based interventions for COVID-19. The study also mentioned a major drawback: maintaining functional bioactivity and increasing the beta glucan yield requires less harmful extraction processes without enzyme and harsh chemical usage. This extraction process is crucial for using beta glucan against COVID-19 [B23].

### AFO-202 Beta Glucan—An Interim Vaccine Substitute

We herein describe a black yeast Aureobasidium pullulans AFO-202 strain (Also referred to as FO-68 [(accession number) FERM BP-19327]) of derived beta glucan that is uniquely secreted as an exopolysaccharide by the black yeast. Therefore, it does not need any kind of extraction-to-purification procedure, which leads to highly pure beta glucan with significant bio-functional activity [B24]. This AFO-202 beta glucan can produce various positive immune responses relevant to COVID-19. It decreased IL-6 levels, which is the most commonly elevated cytokine in a COVID-19 cytokine storm, which is the main mechanism leading to organ damage and mortality. It enhances IFN-γ and sFAS. It is associated with increased production of IL8 which causes the activation, migration and chemotaxis of neutrophils virus cytotoxicity. It decreases CCL2 and CXCL10 levels, thus preventing chemoattraction for monocytes/macrophages, T cells, NK cells and dendritic cells. The prevention of chemoattraction then modulates immune responses. It also increased IL-7 production, leading to mature T cells' survival and development. Activating CD8+ (cytotoxic T cells), CD4+ (mainly Th1 cells) and Treg cells helps balance the regulatory immune response. Activation of B cells by this AFO-202 beta glucan results in the production of virus-specific antibodies [B24,25]. This AFO-202 beta glucan enhances NK cell activity against Leishmania amazonensis infection [B26]. This AFO-202 beta glucan is also present in the inner wall of Candida Albicans, thereby strengthening its role as a PAMP and leading to significant recognition by PRRs. This AFO-202 beta glucan has been continuously consumed since 1996, when the Japanese regulatory authority approved it as a food additive. It has been established as safe and efficacious in several studies, including those with elderly patients [B27]. This AFO-202 beta glucan also helps maintain blood glucose and lipid levels [B28,29], thereby addressing the high risk from co-morbidities like diabetes and cardiac diseases in the pathogenesis of COVID-19.

### AFO 202—as a Wide Spectrum Immune Effector

Oral vaccines generate immunity in gut-associated lymphoid tissue (GALT) that consists of lymph nodes, Peyer's patches (containing 75% of B cells and 20% of T cells) and isolated lymphoid follicles in the gastrointestinal tract (GIT). M cells transport the antigen in the vaccine across the mucosal barrier into Peyer's patches, and the antigen is presented to T cells by antigen-presenting cells (APCs). The CD4+ T cells are activated, which supports germinal centre development, B cell affinity maturation and class-switching to IgA, along with CD40/CD40 ligand interactions and cytokine secretion. The antigen-primed B cells then migrate to distant effector regions, where they differentiate into plasma cells that secrete dimeric or polymeric IgA molecules. These molecules are transported into the intestinal lumen as SIgA, secretory IgA (sIgA) antibodies that prevent attachment and pathogen invasions and neutralize enterotoxins and induce serum IgG antibodies via the vaccine, which act against mucosally and systemically invasive pathogens. Vaccines also activate cell-mediated immune responses (CMI) against intracellular bacteria and viruses along with antibody-dependent cellular cytotoxicity responses [B30, 31].

Following intra-dermal vaccination, immune cells (such as DCs, T lymphocytes, NK cells, macrophages, and mast cells) present in the skin epithelium trigger the skin's inflammation cascade, mainly via Langerhans cells (a specific DC subset that migrates into the lymph node following antigen capture and initiates the adaptive immune response). These cells are stimulated by pathogen-associated molecular patterns (PAMPs) via an array of germline-encoded pattern recognition receptors (PRR), including toll-like receptors (TLR) and langerin (CD207). The skin's resident mast cells induce the innate immune response in the skin by releasing granules containing inflammatory mediators [B31].

The immune system trigger pathways of oral and intra-dermal vaccination depend on components of the reticulo-endothelial system or the mononuclear phagocyte system [B32] employed to access the immune system. Oral vaccines start with mucosal-associated lymphoid tissue (MALT) and GALT, and intra-dermal vaccines start with peripheral lymphoid tissues.

Beta glucans have been suggested to be promising anti-infective vaccine adjuvants, as they alone can stimulate various immune reactions including antibody production without any adverse reactions. Beta glucan has been employed as adjuvants to vaccines against Yersinia ruckeri, though in that care it was not a direct part of the vaccine [B33]. Beta Glucan as adjuvants has been found to enhance immunogenicity of hepatitis B vaccine, influenza vaccine, vaccines against systemic aspergillosis and coccidioidomycosis. The AFO-202 beta glucan has been proven to be a potential immune adjuvant because when it was administered with an avian influenza H5 subtype vaccine, it initiated significantly higher immune responses with higher hemagglutination inhibition (HI) titres and 10-20% ELISA seroconversion [B34]

### EXAMPLES

Hereinafter, the present invention will be described more specifically based on the following literature studies and examples. It should be noted that this embodiment does not limit the present invention.

### Literature Study

### Study 1

### Vulnerable Populations Needing a Continuous Supplementation Approach for Cancer Prevention:

From the above description, we can thus identify subsets of vulnerable populations who are at high risk for either cancer development or who have cancer but need intervention for preventing the cancer progression.

They are:

i. Aged individuals with inflammaging: There is sufficient evidence on how age-related pathologies including cancer, cardiovascular diseases and type 2 diabetes reveal a common inflammatory background, the process termed as inflammaging. Chronic antigen load by infections, cellular senescence, dysregulated DNA damage response, altered gut microbiota, metaflammation and some miRs which are associated with aging also influences the other causative factors for cancer all together influencing and foster inflammaging leading to cancer formation and progression (Leonardi)
ii. People with genetic risk variants for cancer development either per se or due to influence on the immune system [C19]: The association between genes and cancer is well known. For instance, the most commonly mutated gene in all cancers is TP53. Inherited mutations in the BRCA1 and BRCA2 genes are associated with hereditary breast and ovarian cancer syndromes [C19]. Imai et al.'s landmark study is very important in regard to the immune system's weakness and cancer development. They assessed the natural cytotoxic activity of peripheral-blood mononuclear cells by isotope-release assay in 3625 residents of a Japanese population mostly older than 40 years of age between 1986 and 1990. They also conducted an 11-year follow-up survey of the cohort members looking at cancer incidence and death. Their follow-up clearly indicated that medium and high cytotoxic activity of peripheral-blood lymphocytes is associated with reduced cancer risk, whereas low activity is associated with increased cancer risk [C23].
iii. Individuals with lifestyle and metabolic disorders [C10]: For more than 70 years, the association between metabolic disorders such as diabetes and cancer has been hypothesized [C10]. Epidemiological data also show that patients with diabetes have an augmented risk of developing various types of cancers as well as increased mortality. Several pathways have been proposed for the association between diabetes and cancer: i. hyperglycaemia leading to increased cancer risk via augmented oxidative stress and DNA damage, ii. hyperinsulinemia due to exogenous insulin or insulin analogues (this view has been challenged by several studies), and iii. chronic microinflammation with cytokine dysregulation [C10]. Hyperglycaemia of diabetes favours malignant cell growth by providing energy to cancer cells. Increased levels of chronic inflammatory markers, e.g., interleukin (IL)-1β, IL-6, and tumour necrosis factor (TNF)-α have been observed in diabetic patients, which highlights the activation of the immunoresponse in the progression and development of cancer cells. The uncontrolled proinflammatory response environment in diabetes caused by chronic accumulation of glycated biomolecules and advanced glycation end products create a chronic inflammatory state by activation of the transcription factor nuclear factor (NF)-_{κ}B and formation of reactive oxygen species in cells, which promote a tumour-favourable microenvironment and potentially trigger immune system overactivation, culminating in cancer growth [C13,14]. In regard to metabolic syndromes, chronic inflammation and cancer, a chronic and stable background inflammation has been proposed. It is referred to as “hypothalamic microinflammation” [C17] due to the hypothalamus atypically undergoing proinflammatory signalling activation with increase in age and development of metabolic syndrome. This Hypothalamic microinflammation has also been reported to programmatically control whole-body aging [C17]. Since aging is associated with a chronic inflammatory state, correlating negatively with longevity and positively with neurodegenerative diseases, the association between the hypothalamus and microinflammation state leading to cancer has become more evident [C17].
iv. People with immune system weaknesses due to i, ii or iii
v. Cancer patients undergoing chemotherapy, radiotherapy or surgery, which lead to therapy-induced immune dysfunction [C27-29]. Chemotherapy or a chemo- and radiotherapy combination has been reported to significantly delay the immune recovery to pre-treatment baseline levels. Surgery leads to a window of opportunity [C28] that allows the residual cancer cells, including distant metastases, to gain a foothold in the absence of NK cell surveillance.

The way forward would be identifying a continuous supplementation approach as an intervention; this would help maintain the immune system at its proper function constantly, thereby enhancing its immune surveillance and anti-tumour properties, playing a potential role in cancer prevention as well.

### The Vaccine Approach to Cancer

According to the Centers for Disease Control (CDC), USA, “vaccine is a product that stimulates a person's immune system to produce immunity to a specific disease” [C31]. Vaccines in cancer may be therapeutic or preventive. Preventive cancer vaccines include those proteins, peptides, DNA or RNA that could elicit or boost pre-existing antitumour immunity, leading to cancer elimination and production of long-term memory to prevent its recurrence [C32]. Therapeutic cancer vaccines' purpose is to control the cancer burden. Such considered vaccines include autologous patient-derived immune cell vaccines, tumour antigen-expressing recombinant virus vaccines, peptide vaccines, DNA vaccines and heterologous whole-cell vaccines derived from established human tumour cell lines [C33]. While several are in clinical trial, the personalized dendritic cell vaccine sipuleucel-T (Provenge) and the recombinant viral prostate cancer vaccine PSA-TRICOM (Prostvac-VF) are well known vaccines in the pre-approval/authorized approval/late clinical trial stages [C34]. Vaccines are administered often with adjuvants which help to improve poorly immunogenic vaccines” [C35].

Different types of novel adjuvants have been identified and applied with cancer vaccines. They include including inorganic nanoparticles, organic molecules and polymers [C36]. Pathogens stimulate a “danger sensing” signal via pathogen-associated molecular patterns (PAMPs). Inorganic nanoparticle-based adjuvants work by acting like PAMPs, stimulating the anti-tumour immunity. Organic molecule-based adjuvants include small molecule-based ones such as modified PAMPs, new ligands for PPRs, etc. Agonists of the toll-like receptor family, which are type I transmembrane proteins that regulate the innate and adaptive immunoresponses [C36]; and agonists of STING (stimulator of interferon genes) [C37] are all examples of organic adjuvants. Polymer-based adjuvants both help in drug delivery and act as PAMPs for immune system activation.

However, a question arises: is there a nutrition-based supplementation which can act as a potential vaccine adjuvant to help in the ongoing fight against cancer, both preventive and therapeutic?

### The Beta Glucan Vaccine Adjuvant (B-VACCIEN) Approach to Conquer Cancer

Beta-glucans are naturally occurring polysaccharides as constituents of the cell walls of certain bacteria and fungi [C38]. Yeast derived 1,3-1,6 beta glucans are reported to have more potent biological response modifier effects [C39]. “An immunomodulator is defined as the substance capable of interacting with the immune system resulting in up- or down-regulating specific parts of the immune response” [C40]. Immunomodulators comprise of an array of synthetic, natural and recombinant molecules. Natural molecules such as those found in curcumin, thyme, bay leaf, resveratrol, ellagic acid, ginseng, echinacea, aloe vera, astragalus, goldenseal, flavonoids and essential oils have all been studied for their immunomodulation properties as nutritional supplements. However, direct comparison studies of individual immunomodulators have been extremely limited. Vetvcika note that with more than 20,000 published studies, glucan gained the best position among other immunomodulators [C40]. Glucans are biological response modifiers which have significant effects on various branches of the immune system. Glucans are recognized by pattern recognising receptors present on the membranes of cells such as macrophages, monocytes, dendritic cells and NK cells, with the key receptors being Dectin-1 and CR3 (CD11b/CD18). Additional receptors are Toll-2, lactosylceramides and the scavenger receptor family [C40].

In terms of cancer-immunity, Beta-glucans have been shown to play pivotal roles as increasing resistance to infection (particularly of importance in virus associated cancers), anti-tumor effects by activating the adaptive and innate arms of the immune system, stimulating immune cells like leukocytes, T helpers, and NK cells and anti-coagulant effects (Chaichian, 2020). Beta glucans activate early innate reactions by acting as PAMPs. Glucan-activated B cells have been shown to secrete pro-inflammatory lymphokines such as interleukin-8 by involvement of several molecules such as Dectin-1 receptors, mitogen-activated protein kinase (MAPK) and the transcription factors NF-_{κ}B and AP-1. Beta glucans have been shown to be strong activators of cellular immunity. The anti-infection effects of beta glucan have been demonstrated against infections such as Leishmania major, L. donovani, Candida albicans, Toxoplasma gondii, Streptococcus suis, Plasmodium berghei, Staphylococcus aureus, Escherichia coli, Mesocestoides corti, Trypanosoma cruzi, Eimeria vermiformis, and Bacillus anthraci. Numerous animal and human studies have proven the anti-tumour effects of beta glucans against a wide variety of tumours [C40]. Recent studies prove that beta glucans have a strong synergy with the antibodies which naturally occur in cancer, as well [C40].

In regard to adjuvant immunotherapy for cancer, both dendritic cell priming and check-point inhibitor blockades have been shown to be required for immunotherapy [C41]. Beta glucans serve as an ideal candidate, as they have both dendritic cell priming and also potentiate antibodies against immune checkpoint molecules [C39]. A combination therapy using β-glucan and mAbs targeting immune checkpoint molecules such as PD-1 and PD-L1 has been investigated in preclinical models with promising antitumour efficacy, and is in the process of being translated into a phase I clinical trial [C39]. Trained innate immunity (TRIM) is innate immune system memory induced by modulation of mature myeloid cells or their bone marrow progenitors. This process helps mediate sustained increased responsiveness to secondary challenges. It has been reported that anti-tumour immunity can be enhanced through induction of trained immunity [C42].

It is worthwhile to note that beta glucans are effective inducers of TRIM, specifically via epigenetically reprogramming the innate immune cells at the level of bone marrow (central TRIM) as well as peripheral TRIM [C43,44]. Vetvicka and Vetvickova [C45] note that highly purified and active glucans have significant pleiotropic effects in cancer. Cancer cell resistance is an important hurdle in anti-cancer therapies. Beta Glucans are potential candidates for overcoming treatment resistance in cancer. This has been proven in treatment-resistant Lewis lung carcinoma cell line (LL/2) cells in which a Candida cell wall beta-glucan showed a significant cytotoxic effect on both the parent cell line and cancer stem cells derived from the cell line (Sadeghi).

### Chronic Micro-Inflammation, Cancer and Beta-Glucans

Chronic Inflammation pre-disposing to cancer has been established with sufficient evidence. Underlying infection or inflammation have been linked to 25% of all cancer cases. Any unresolved inflammation on account of any failure in the precise control of the immune response can continues to disturb the cellular microenvironment, leading to alterations in cancer-related genes and posttranslational modification in key cell signalling proteins involved in cell cycle, DNA repair and apoptosis. Identification of mononuclear inflammatory cells (MICs) in close association with areas of hyperplasia and atypia even at very early stages of tumour development, further support the concept that inflammation is a major driving force that contributes to tumor initiation and/or initial tumoral progression. Upregulation of non-specific pro-inflammatory cytokines (interferon-y, tumor necrosis factor (TNF), interleukin (IL)-1α/β or IL-6) by immune cells such as macrophages, mast cells and neutrophils have been shown to promote tumour development (Neiro). The inflammatory processes elicited by the cancer itself are likely to be involved in their progression.

Inflammation is also the common mechanism of action for numerous cancer risk factors such as infection, obesity, tobacco smoking, alcohol consumption, exposure to microparticles, dysbiosis and chronic inflammatory diseases including pancreatitis and colitis. Consumption of certain anti-inflammatory drugs, including aspirin, have also been shown to significantly reduce cancer risk. Preventing or reversing inflammation has been suggested as an important approach to cancer control (Todoric).

Chronic-microinflammation culminating in cancer also needs attention on metabolic disorders such as diabetes and cancer formation. With Several pathways having been proposed for the association between diabetes and cancer such as hyperglycaemia leading to increased cancer risk via augmented oxidative stress and DNA damage, hyperinsulinemia, the chronic microinflammation with cytokine dysregulation needs specific attention. The uncontrolled proinflammatory response environment in diabetes caused by chronic accumulation of glycated biomolecules and advanced glycation end products create a chronic inflammatory state by activation of the transcription factor nuclear factor (NF)-_{κ}B and formation of reactive oxygen species in cells, which promote a tumour-favourable microenvironment and potentially trigger immune system overactivation, culminating in cancer growth.

Further on chronic inflammation and cancer, a chronic and stable background inflammation has been proposed referred to as “hypothalamic microinflammation” [C17] which is the hypothalamus atypically undergoing proinflammatory signalling activation with increase in age and development of metabolic syndrome Beta Glucans especially yeast derived beta glucans help in combatting chronic microinflammation contributing to cancer-preventive response apart from their metabolic balancing activities which also adds to their effects in cancer prevention. In a study of a yeast derived beta-glucan, the antioxidant activity by H2O2 scavenging, in vivo anti-inflammatory potential in terms of myeloperoxidase activity and reduction in MDA and NO were all demonstrated (Bacha). In another study, regular intake of Beta Glucan was demonstrated to have an anti-inflammatory effect, by acting on IL-6 a pleiotropic cytokine that plays pivotal role in acute phase responses in the balancing of the pro and anti-inflammatory pathways (Barera). An AFO-202 biological response modifier glucan (BRMG) derived from a black yeast called the Aureobasidium pullulans AFO-202 strain (also referred to as FO-68 [(accession number) FERM BP-19327])with high purity and functionality which is water-soluble beta glucan and in human consumption for several decades [C46] can serve as a potential Beta-glucan Vaccine Adjuvant approach to Conquer Cancer through Immune-ENhancement; the B-VACCIEN adjuvant approach to cancer for its following characteristics and attributes. AFO-202 beta glucan has been shown to be beneficial in maintaining blood glucose levels and lipid levels in the normal range in several human studies [C47-49], thereby helping to prevent the metabolic micro and chronic inflammation leading to cancer. AFO-202 beta glucan has been proven to stimulate the production of interleukin-8 (IL-8) or soluble Fas (sFas), but not that of IL-1beta, IL-6, interferon-gamma (IFNG), tumour necrosis factor-alpha (TNF-alpha) or soluble Fas ligand (sFasL) [C46].

IL8 has anti-inflammatory activity and helps in recruitment of T cells. It also enhances the metabolism of ROS (reactive oxygen species). It serves as a barrier against invading microorganisms and airway epithelial release of IL-8 contributes to host's immune defense by promoting neutrophil chemotaxis (Qazi). Tumours have been shown to express Fas ligand (FasL) and down-regulate Fas to escape from host immune surveillance. Elevated serum sFasL levels has been associated with a disease progression (Kozlowski) Cytokines such as IL1 and IL6 are inflammatory cytokines, especially IL-1, IL-4 and IL-6, secreted by immune cells in the tumour microenvironment observed in large range of solid tumours, whose receptors' expression by the cancer cells help with their immune evasion (Setrerrahmane). Since IL-6 promotes tumour growth and its elevated serum levels and increased expression in tumour tissue are negative prognostic marker for cancer patients' survival (Chonovoc).

Though IFNG has been long considered as a central player in antitumor immunity, it also has protumorigenic roles. IFNG-mediated activation of the non-classical MHC class Ia genes has been shown to help melanoma cells evasion from CTL mediated cytolysis in turn leading to clinical failure of melanoma peptide vaccines. IFNG also is associated with influx of monocytic and granulocytic myeloid-derived suppressor cells to the tumor microenvironment which leads to—Suppression of anticancer T cell response. IFNG-induced PD-L1/2 ligands on the cancer cells causing them to bind to their immune inhibitory receptor PD-1 which suppresses the immune effector activities of T cells and NK cells promoting cancer progression (Zaidi).

TNF-α, primarily secreted by tumor-associated macrophages initiates chronic inflammation. TNF-α also has dual roles wherein it causes tumour cell apoptosis when administered in high doses but long-term low dose administration has been shown to accelerate tumour invasion and metastasis. TNF-α also induces the expression of angiogenic factors, promoting tumor angiogenesis and accelerating tumor metastasis by upregulation of tumor-associated calcium signal transduction protein (TROP)-2 via the ERK1/2 signaling pathway (Zhao). Thus, the AFO-202 Beta Glucan by balanced activation of anti-cancer cytokines and suppression of pro-tumorigenic cytokines could play a key role to prevent the cytokine imbalance inflammation induced by chemotherapy or other cancer therapies and help in anti-cancer therapeutics. This improves cancer prevention and therapeutics [C27-29].

It has been reported that the Dectin-1 based recognition of tumour cells orchestrates innate immune cells' anti-tumour responses [C50]. The key receptor by which the AFO-202 exerts its biological response modifying effects is Dectin-1 [C46]. This AFO-202 beta glucan has been shown to help against infections. For example, it enhances immunity against L. amazonensis and malaria by increasing NK activity and cellular immunity, extending anti-cancer potential by fighting against infections [C51]. Already, the vaccine adjuvant effects of AFO-202 beta glucan have been reported as a potential effector enhancing immune response to the avian influenza A H5N1 and H5N2 vaccines [C52].

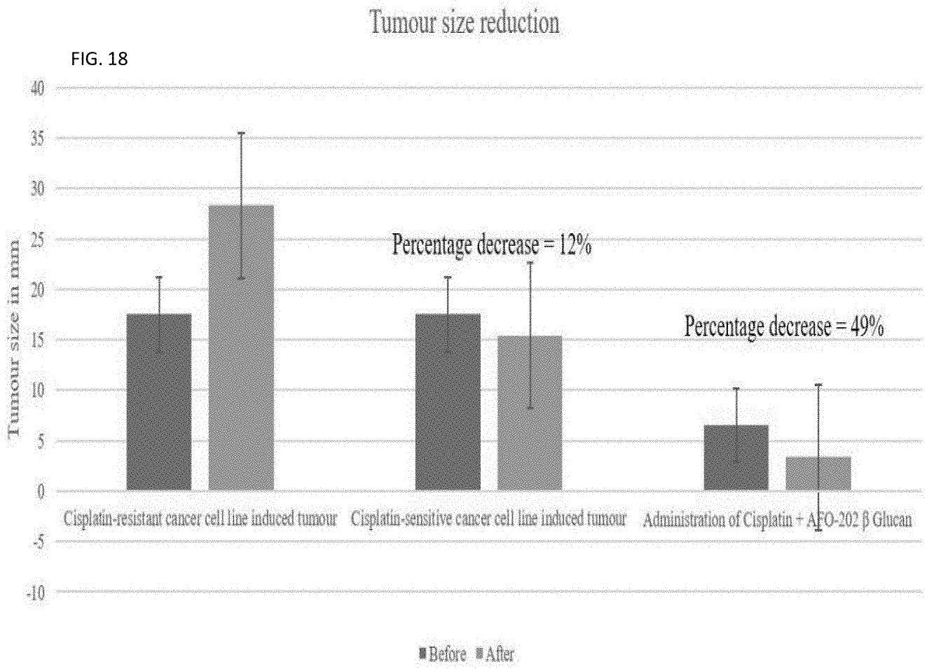

In animal models of tumour implant, the comparative anti-tumour effect of AFO-202-derived beta glucan has been significantly higher than other types of glucans [C53]. When mouse models of tumours were administered AFO-202 beta glucan, the immune profile increased and maintained to normal levels similar to control groups without chemotherapy [C54]. The percentage of tumour size decrease after cisplatin chemotherapy in the study by Ma et al. [C55] was 12%, compared to 49% in a study in which AFO-202 beta glucan was administered [C56] along with cisplatin chemotherapy (FIG. 18). Eleven healthy human volunteers consumed 15 g of AFO-202 beta glucan orally 3 times a day for a month, and the cytotoxic activity of the NK cells from their peripheral blood-derived mononuclear cells was assessed against K562 cells derived from human stomach cancer cell line.

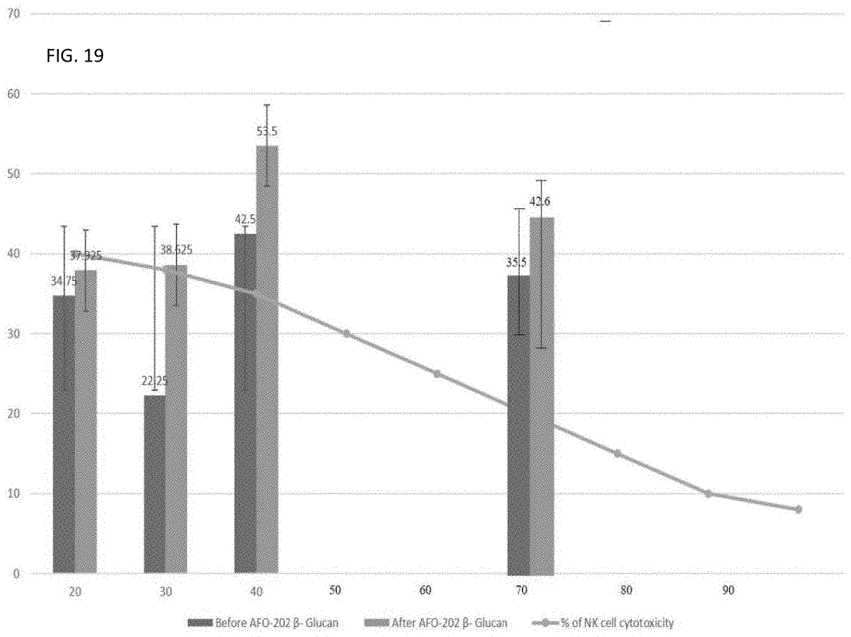

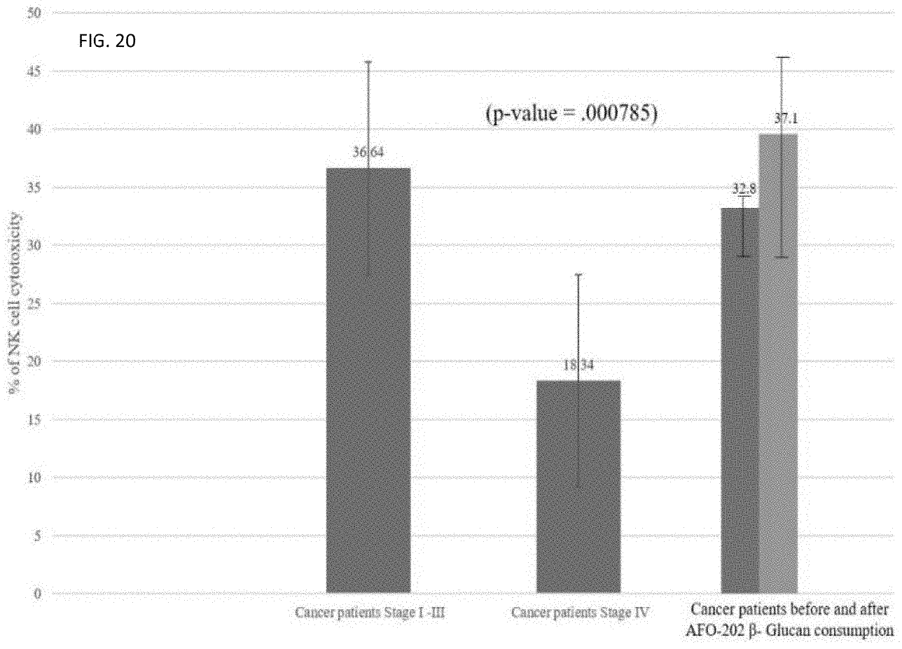

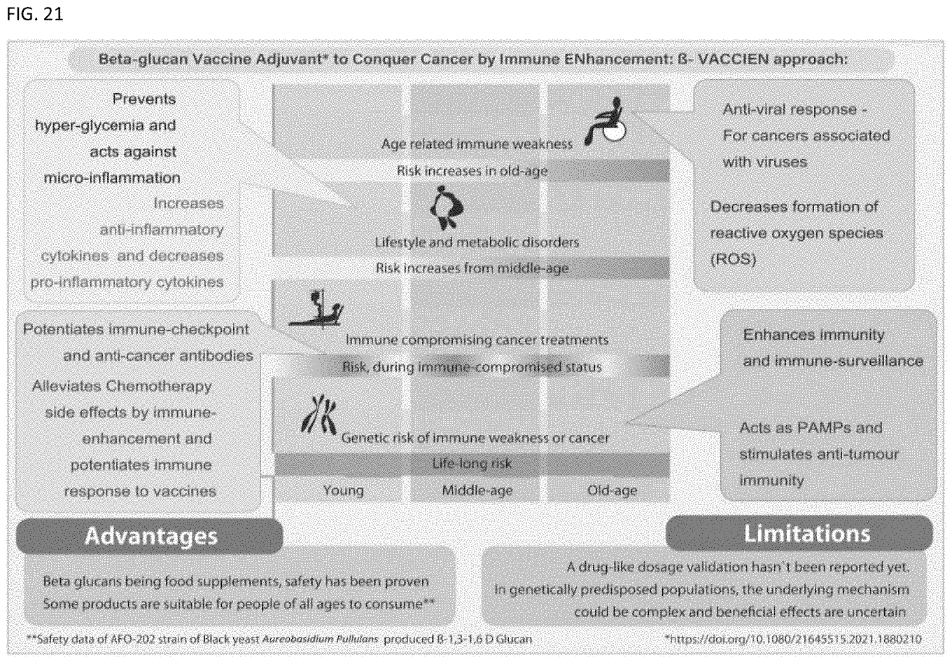

The rate of increase of the cytotoxic activity was 90.9% [C54]. When this data of increase in NK cell cytotoxicity in healthy individuals was correlated with historical data from the literature [C57], it was found that across all ages, the consumption of AFO-202 beta glucan [C54] significantly increases NK cell cytotoxicity (p-value=0.031599; FIG. 19). In another study of AFO-202 derived beta glucan in cancer patients (n=35) conducted to examine the immunostimulatory effects of oral consumption of 15 g of AFO-202 derived beta glucan 3 times a day for 3 months, the increase in NK cell activity in elderly cancer patients increased from 32.8% to 37.1% [C58], which when correlated with historical data from the literature [C59] is significantly higher (p-value=0.000785; see FIG. 20).

An illustration on how beta glucan supplementation can contribute to anti-tumour immunity and alleviate cancer therapy-induced side effects in specific populations who are predisposed to develop an immune system weakness is given in FIG. 21.

The evolution of the immune system takes an uphill curve in regard to cancer with contributions from viruses and chronic inflammation. With lifestyle and metabolic disorders having become major healthcare-related issues in the latter half of the past century, and with micro-inflammation serving as the underlying mechanism leading to cancer in such individuals, a senile immune system weakness or inflammaging is unavoidable. It may occur in any individual, even though they may not have chronic inflammation. All the above culminate in a dent in the immune system whose address requires a holistic approach that could potentially act against virus, infection, inflammation and metabolic disorders, in addition to acting as a continuous supportive mechanism preventing immune surveillance system weakening. Apart from these factors, the genetic component of immune system weakness or genetically prone cancers may further add fuel to the fire of immune system weakness. Genetics also needs to be addressed—in these individuals, the time at which immune system weakness develops or the aggressiveness of the cancer which may occur are unknown.

A continuous vaccine adjuvant approach could use food supplements such as beta glucan. Although we are not sure whether the immunoenhancement will completely address any cancer already formed, we feel it will definitely be a potential strategy to address the periodic or intermittent jeopardy to the immune system. The window period of immune system weakness after surgery, as well as the immune system weakness induced by chemo- or radiotherapy, need definite examination; immunosuppression is considered a major reason for treatment failure in cancer [C60]. Treatment strategies to overcome immune system weakness after cancer therapies requires large-scale translational and clinical research. We hope such research will yield some insights into how chemotherapy, surgery or radiotherapy-related cancer treatments can be supplemented by the beta glucan vaccine adjuvant approach to alleviate side effects. This goal can be achieved by effectively engaging the immune system to reduce cancer-adverse reaction-related morbidity and mortality.

It is significant to note that there is an ongoing randomized phase I/II trial studying the side effects and best dose of OPT-821 (saponin-based immunoadjuvant OBI-821) with vaccine therapy when given together with beta-glucan and how well the regimen works in treating younger patients with neuroblastoma (https://www.cancer.gov/about-cancer/treatment/clinical-trials/search/v?id=NCI-2009-01362&r=1)

### Study 2

A literature search on mortality-related comorbid conditions was performed. For those conditions, we analyzed the pro-inflammatory cytokines, which could cause the draining of the immune reservoir. We also analyzed the immune markers necessary for the defense mechanism/immune surveillance against COVID-19, especially through simple means including immune enhancing nutritional supplement consumption, and we suggest strategies to combat COVID-19. Major comorbid conditions associated with increased mortality include cardiovascular disease (CVD), diabetes, being immunocompromised by cancer, and severe kidney disease with a senile immune system. Consumption of Aureobasidium pullulans strain (AFO 202) beta 1,3-1,6 glucan supported enhanced IL-8, sFAS macrophage activity, and NK cells' cytotoxicity, which are major defense mechanisms against viral infection.

### Conclusion

People with co-morbid conditions who are more prone to COVID-19-related deaths due to immune dysregulation are likely to benefit from consuming nutritional supplements that enhance the immune system. We recommend clinical studies to validate AFO 202 beta glucan in COVID-19 patients to prove its efficacy in overcoming a hyper-inflammation status, thus reducing the mortality, until a definite vaccine is made available.

The immune system is a double-edged sword (A67) that has balance between its primary activity of defense against foreign pathogens, oncogenesis, and circulating cancer cells while maintaining their limitations from overacting and ending up in a hyperinflammatory status, which leads to severe cytokine storm in COVID-19 patients. Although specific targeted molecules and agents that can act on each step of such an immune system may be efficacious for providing beneficial effects they continue to have adverse reactions. Given this background, broadly acting non-harmful strategies are currently considered essential, given that the lack of a definitive vaccine to “complex” the COVID-19 pandemic is threatening vulnerable populations with immune dysregulation. Through this analysis, we have found that a proven primary immune defense-improving and immune-modulation oriented nutritional supplement such as the AFO 202 beta glucan could be tried in these patients in a multi-centric study to prove its efficacy. Its consumption as a food supplement has proven safe for more than two decades.

### Example A

Sixteen human subjects are orally ingested with the additive-free jelly (n=8) and the FO-68-derived β-1,3-1,6 glucan-added jelly (n=8) for 3 weeks. After that, peripheral blood is collected from the veins of all subjects, and the amount of various cytokines production, the number of immune cells, and the blood coagulability/thrombogenicity parameters are measured.

The following results are obtained with respect to the increase/decrease in various cytokine production, the number of immune cells and blood coagulability of the glucan-added jelly ingested group relative to the additive-free jelly ingested group.

TABLE 1

| | Relative | | Relative |
|---|---|---|---|
| Measurement | increase ↑/ | Measurement | increase ↑/ |
| item | decrease ↓ | item | decrease ↓ |
| IL-1β | ↑ | IFN-α | ↑ |
| IL-6 | ↓ | IFN-β | ↑ |
| IL-12 | ↓ | IFN-ε | ↑ |
| IFN-γ | ↑ | IFN-ω | ↑ |
| TNF-α | ↓ | IFN-∪ | ↑ |
| | | IL-7 | ↑ |
| NKcell | ↑ | Fibrin | ↑ |
| Th2cell | ↓ | D-dimer | ↓ |
| Th2cell | ↑ | Thronbin | ↓ |
| CD8-Tcell | ↓ | | |
| CD4-Tcell | ↑ | | |
| B-cell | ↑ | | |

### Example 1

### F2S Study in SD Rats

### Methods:

Nichi Glucan AFO-202 was administered to 6 SD Male Rats and compared with control (administration of water for injection). The Rats were sacrificed after 15 days. Lymphocyte-to-CRP ratio: Lymphocyte count (number/μL)/CRP (mg/dL) and Neutrophil-to-lymphocyte ratio: the number of neutrophils and lymphocytes taken per 103/μL (NLR) were analyzed.

### Results:

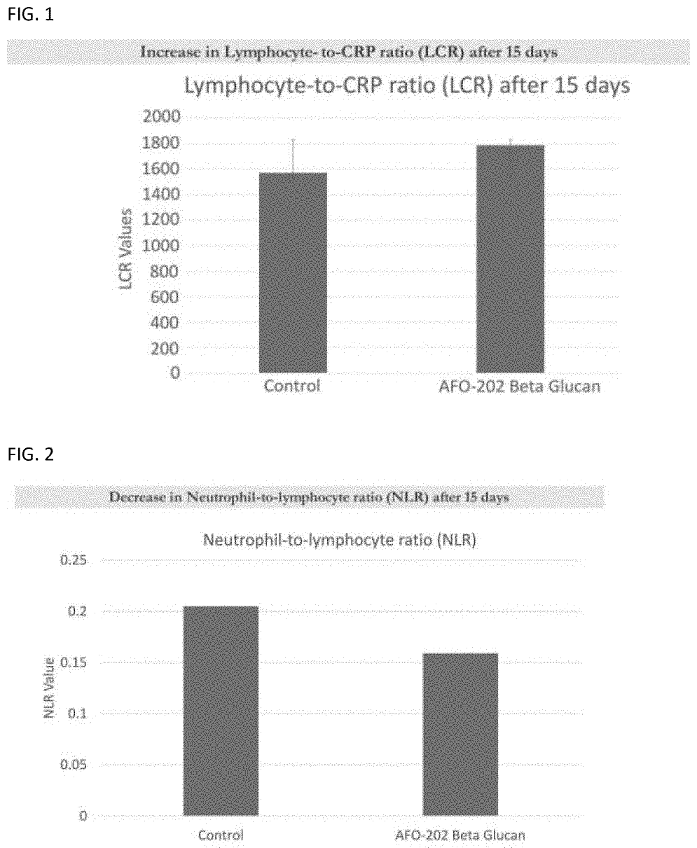

### See FIGS. 1-2.

AFO-202 Beta glucan showed an increase in LCR after 15 days of administration.
AFO-202 Beta glucan showed a decrease in NLR after 15 days of administration.

### Discussion:

Increased NLR levels and low LCR levels reflect an enhanced inflammatory process suggesting a poor prognosis in cancer and infections.

Therefore, AFO-202 beta glucan leading to increase in LCR levels and decrease in NLR levels is advantageous in terms of anti-cancer and anti-microbial effects.

### Example 2

Our group has initiated a pilot study in healthy volunteers, (Men aged between 40˜60) on evaluation of biomarkers relevant to thrombogenicity apart from immune enhancement and immune modulation after AFO-202 beta glucan consumption and the interim results are encouraging.

### F4S Study in Healthy Male Volunteers:

### Methods:

Eight male healthy volunteers ranging in age from 40 to 60 (six volunteers in their 40s, one volunteer in his 50, one volunteer in his 60s) took part in this clinical trial. Volunteers were divided into two groups.

Group A: (n=4): Consumption of AFO-202 Beta Glucan for 21 days
Group B: (n=4): Consumption of AFO-202 Beta Glucan for 35 days

### Results:

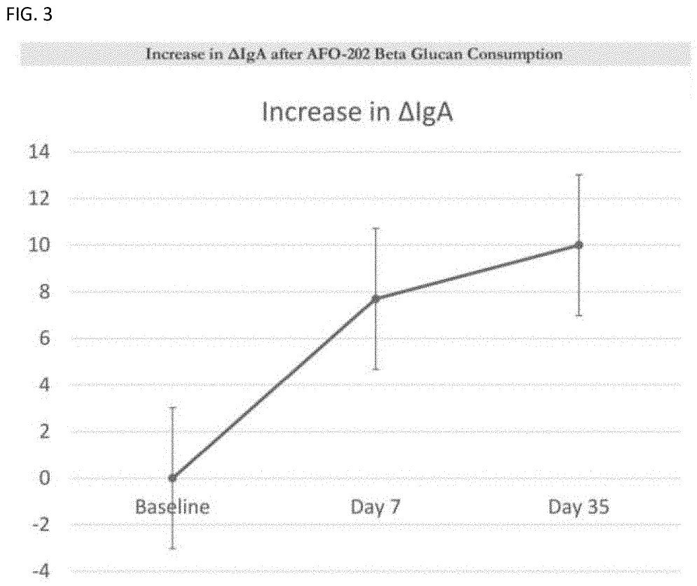

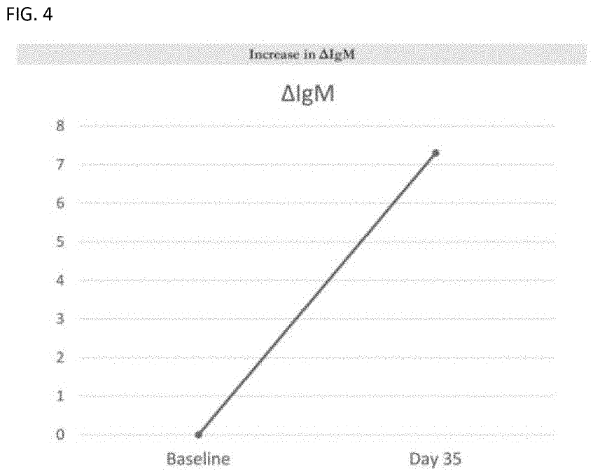

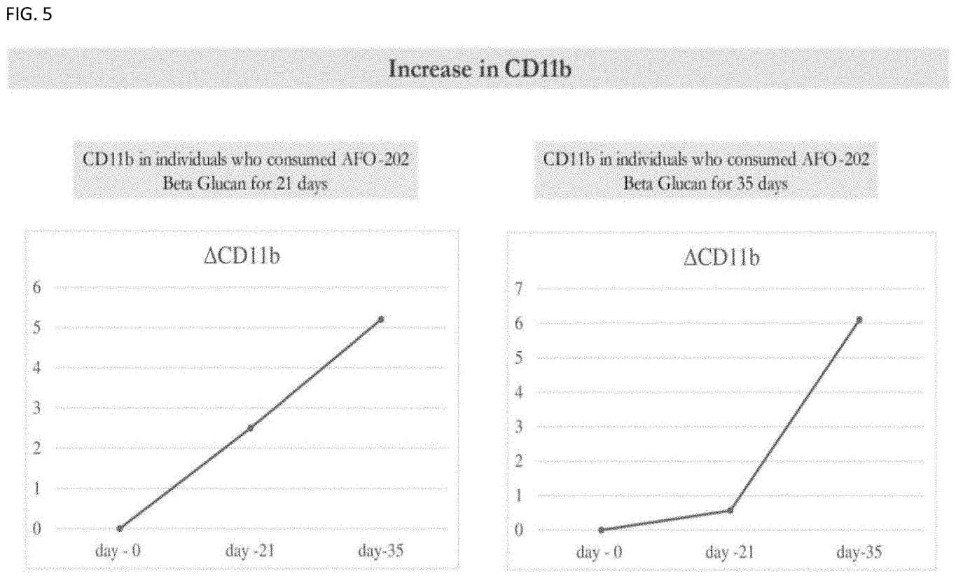

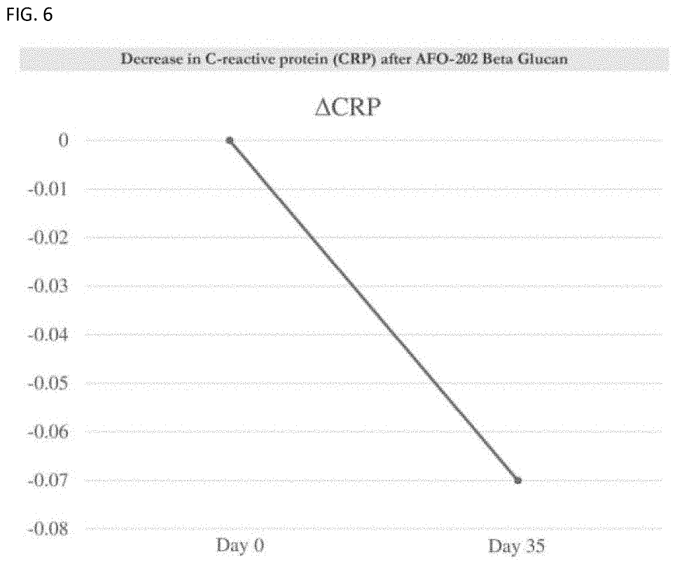

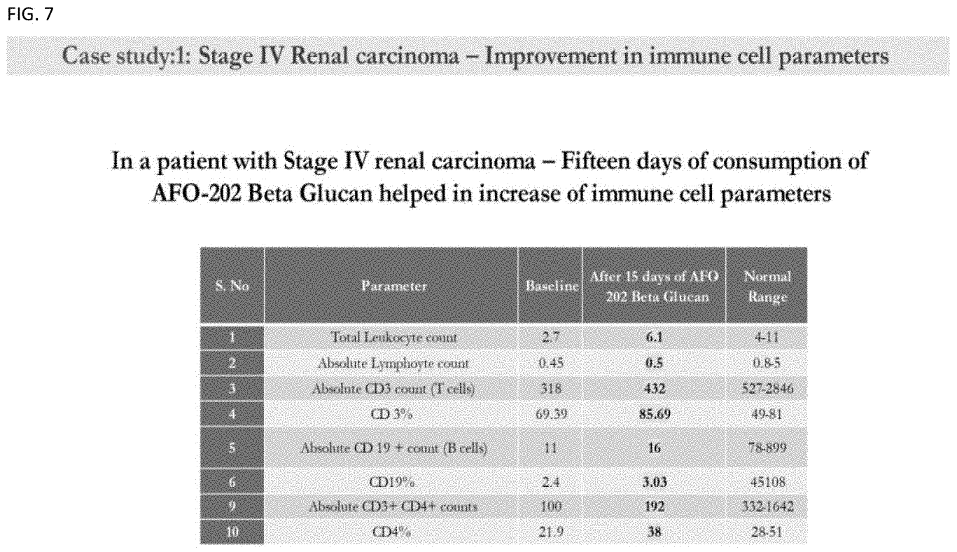

### See FIGS. 3-6.

### AFO-202 Beta Glucan Consumption Lead to

Increase in ΔIgA antibody
Increase in ΔIgM antibody
Increase in CD11b
Decrease in C-reactive protein (CRP)

### Discussion:

IgA antibody plays a key role in the immune defense of mucous membrane. IgA antibodies are superior in killing cancer cells and invading pathogens. Increase in IgA by AFO-202 beta glucan helps to enhance the defense of mucous membranes and body surface lining where the viruses and microbes gain entry.

IgM antibody is the first set of antibodies produced in response to an infection. Increase in IgM antibody levels is an indicator of enhanced immune defense.

Integrin CD11b activation drives innate immunity to resolve infections and tumour growth. Therefore, increase in CD11b by AFO-202 beta glucan is helpful.

High levels of CRP is a poor prognostic factor indicating inflammation in the body. Therefore, decrease in CRP by AFO-202 beta glucan is advantageous.

### Example 3

### F8S (Mitochondria, IL6 and SARS-CoV) Study

### Methods:

HeLa cells (50×106 cells/dish) were diluted 100-fold with β-glucan AFO-202 (BG-A), NN-163 (BG-B) and NN-163 (BG-C) to a final concentration of 1 μg/mL and incubated (stimulated) in a CO2 incubator for 24 h. After 24 h, the cells were collected and washed three times with PBS, total RNA was extracted and cDNA was synthesized using the RNA as a template.
Specific primers for mitochondrial ATPase, mitochondrial DNA (ND1), mitochondrial DNA (ND5), IL-6, ACE2 receptor (ACE2R), CD13 and PDL-1 were then prepared for each. Specific primers for mitochondrial ATPase, mitochondrial DNA (ND1), mitochondrial DNA (ND5), IL-6, ACE2 receptor (ACE2R), CD13 and PDL-1 were then prepared.
Primer and cDNA were added to the PCR reaction system along with a reagent (intercalator: TB Green) that fluoresces by binding to the double-stranded DNA in the PCR reaction system, and the amount of amplified product produced was monitored in real time by detecting the intensity of the fluorescence.
Data were compared using the ΔΔCt method. β-actin was used as a control (control mRNA gene).

### Results:

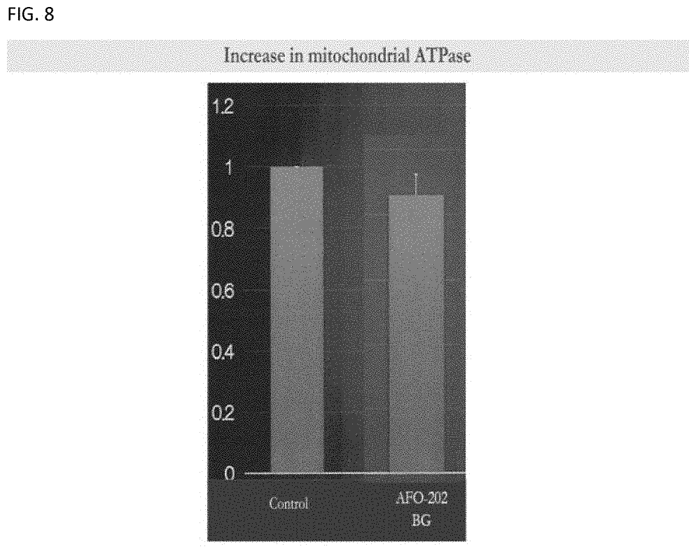

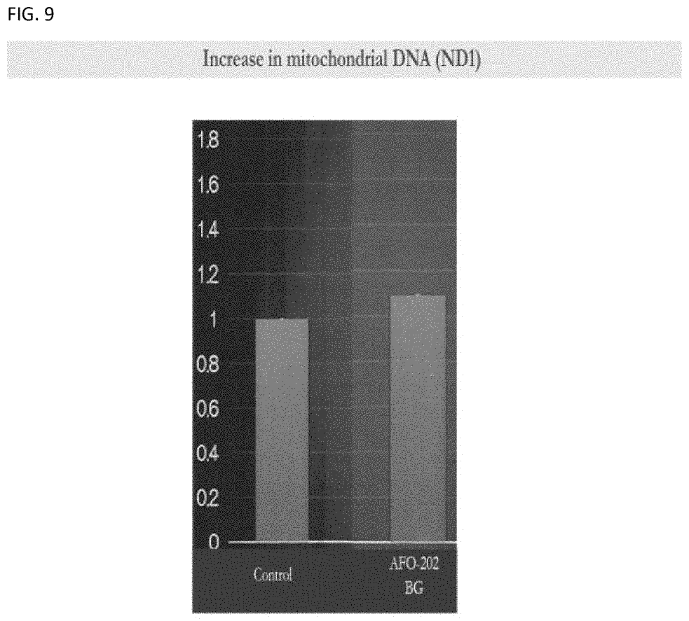

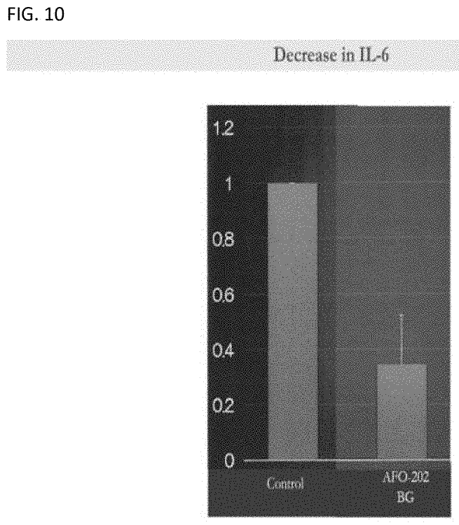

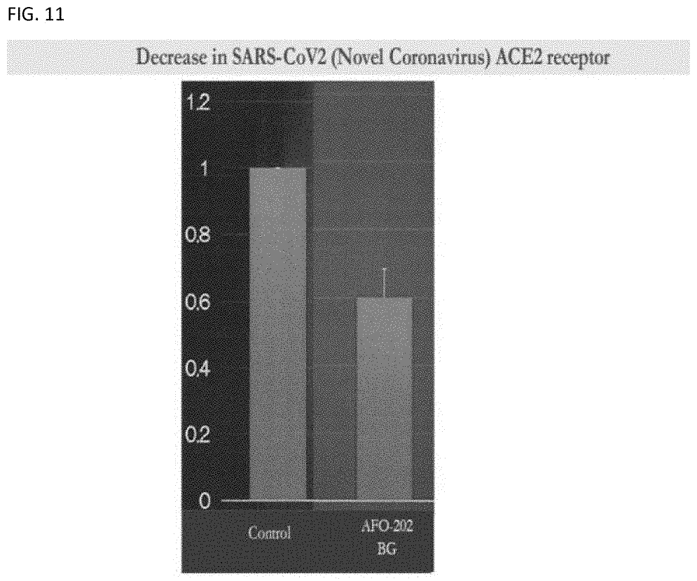

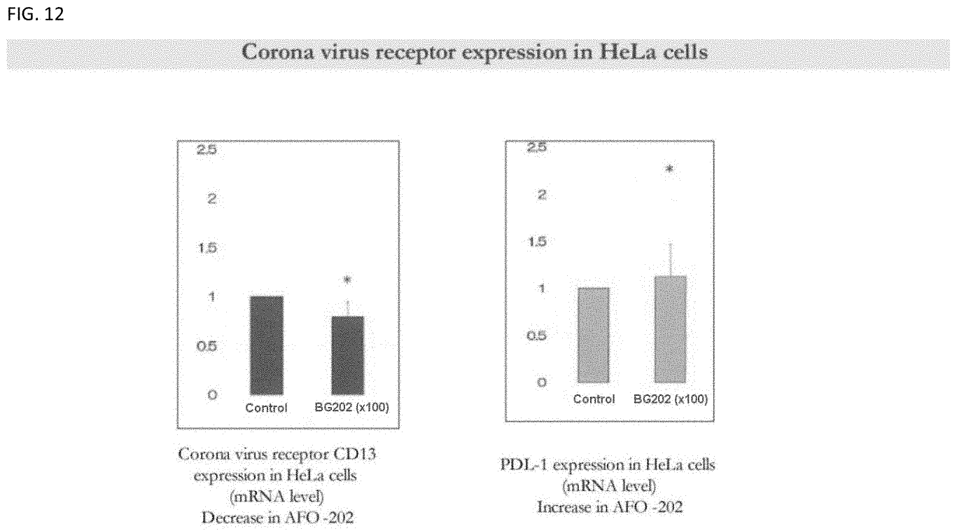

### See FIGS. 8-12.

### AFO 202-Beta Glucan,

increased mitochondrial ATPase and mitochondrial DNA (ND1)
AFO 202 decreased IL-6
Decreases SARS-CoV2 (Novel Coronavirus) ACE2 receptor expression
Decrease in mRNA levels of Corona virus receptor CD13 and PDL-1 expression in HeLa cells

### Discussion:

Mitochondria are considered as the central hub of energy driving the immune system. Aerobic glycolysis is the preferred metabolic pathway of activated immune cells. Therefore AFO 202 glucan can enhance the mitochondrial function thereby enhancing the immune system.

IL-6 is a key cytokine promptly produced in response to tissue infection and injury. Dysregulated continuous synthesis of IL-6 plays a pathological effect in chronic inflammation and is responsible for the cytokine storm causing organ damage in infections like COVID-19. Therefore, reduction of IL-6 AFO-202 beta glucan is beneficial.

Angiotensin converting enzyme 2 (ACE2) has been identified as the receptor to which SARS-CoV2 binds and gains entry into human epithelial tissues such as the airway epithelium and intestinal epithelium. Cells that express little ACE2 were poorly infected with SARS-CoV2. Hence, AFO-202 beta glucan decreasing ACE2 receptor expression will be of potential in preventing COVID-19 infection.

Decrease in Corona virus receptor CD13 and PDL-1 expression will help as a protective effect against coronavirus infections.

### Example 4

### F17S (Antibodies) Study

### Methods:

The levels of anti-CD69 and anti-Candida antibodies were assayed using enzyme-linked immunosorbent assay (ELISA kit details) under the manufacturer's instructions.

### Results:

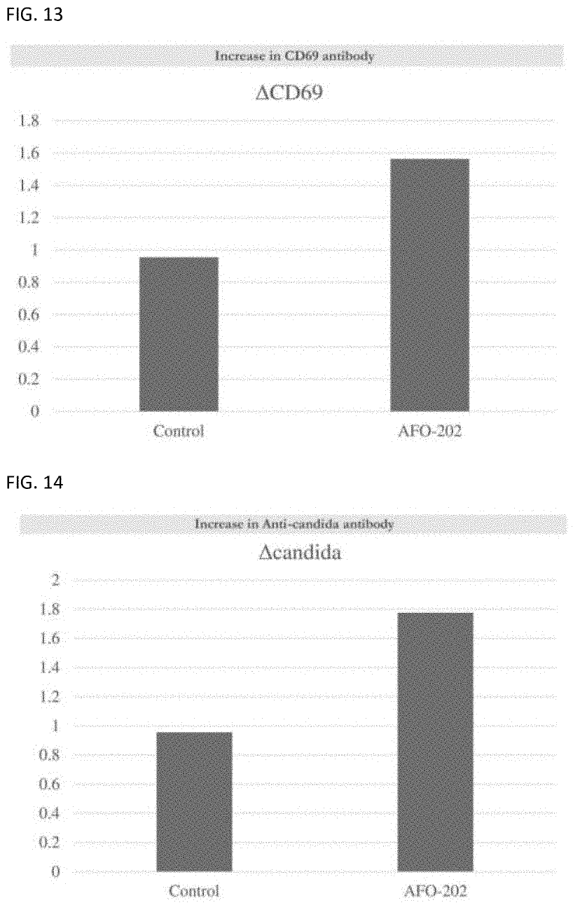

### See FIGS. 13-14.

### AFO 202-Beta Glucan Led to

Increase in CD69 antibody
Increase in Anti-candida antibody

### Discussion:

Anti-CD69 antibody is a potential therapeutic agent against tumour acting via NK cells. Therefore, increase of CD 69 antibody by AFO-202 beta glucan will help in immune activity against cancer.

Increase in Anti-candida antibody response will be helpful in exerting anti-fungal immunity.

### Example 5

### F20S (ATP Synthesis) Study

### Methods:

The ATP hydrolysis rates in Staphylococcus aureus bacterial cells were determined using phenol red to detect proton consumption during ATP hydrolysis.

### Results:

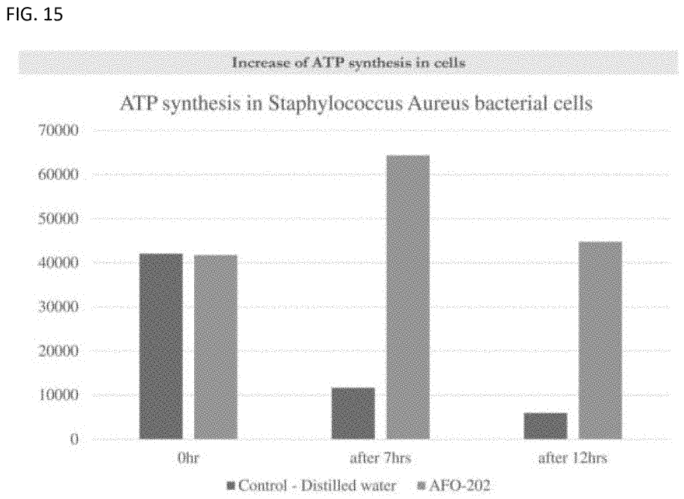

### See FIG. 15.

AFO-202 beta glucan led to increase in ATP synthesis of the bacterial cells.

### Discussion:

Cellular metabolism is important in terms of regulating immune response. Therefore, an increase in ATP synthesis by AFO-202 Beta glucan will help in an effective immune response.

### Example 5

### Cancer Patients—Two Case Studies

### Example 5-1

### Case Study 1: Stage IV Renal Carcinoma—Improvement in Immune Cell Parameters

### Methods:

In a patient with Stage IV renal carcinoma—Fifteen days of consumption of AFO-202 Beta Glucan helped in increase of immune cell parameters

### Results:

### See FIG. 8.

AFO-202 Beta Glucan helped in increase of T cell and B cell count.

### Discussion:

Enhancement of T cell and B cell response helps to increase anti-tumour immunity. Therefore AFO-202 beta glucan helps in anti-tumour response.

### Example 5-2

### Case Study 2: Stage IV Burkitt Lymphoma—Alleviation of Chemotherapy Side Effects

In a patient with Burkitt's Lymphoma Stage IV, continuous consumption of AFO-202 Beta Glucan (6 g prior to and during chemotherapy; 3 g in between cycles of chemotherapy) has helped in alleviating the side effects of six cycles of chemotherapy with R-CHOP regimen (rituximab, cyclophosphamide, doxorubicin, vincristine, and prednisone).There was not even a single episode of vomiting and the patient is responding well to chemotherapy.

### Post Chemo—PET CT after AFO-202 Beta Glucan Consumption

Compared to previous PET/CT done elsewhere, there is:

1. Regression in size3, number and metabolic activity of omental, peritoneal and mesenteric deposits.

2. Resolution of metabolic activity with regression in size of mesenteric nodes, Resolution of bilateral external iliac and right internal iliac nodes.

3. Resolution of hypermetabolic deposits in ileum, jejunum, adrenals, intramuscular regions and bone marrow.

4. Anastomotic site is unremarkable.

5. No demonstrable metabolically active disease elsewhere in the whole body survey.

Overall imaging features are suggestive of partial response to therapy.

### Discussion:

AFO-202 beta glucan helps in anti-tumour response of resolution of metastatic deposits and also helps in alleviating the side effects of chemotherapy such as nausea.

### Example 6

### Covid-19 Patients—Case Studies

### Inclusion Criteria:

1. Adult subjects between 18 and 65 years (both ages and sexes inclusive) who are confirmed to be positive for SARS-CoV2 by way of RT-PCR in a laboratory

2. Subjects with co-morbidities can be included. To be analyzed as cohort

3. Subjects who are found to be Covid19 positive requiring hospitalization. (symptomatic or asymptomatic)

4. Subject and LAR who is willing to give informed consent for participation, able to comprehend and understand the responsibilities during treatment period.

5. Subjects who are willing not to participate in any other clinical trial during participation in the current trial.

### Exclusion Criteria:

1. Subjects who have previously been infected with SARS-CoV2 (symptomatic or asymptomatic) and recovered.

2. Subjects who are known to be HIV, HBV, HCV positive.

3. Subjects who have clinically abnormal renal or hepatic function values that are 3× times normal upper limit or in the opinion of the Investigator would impact the objectives of the study.

4. Subjects with complete cancer remission less than 3 years prior to the date of screening.

5. Subjects who have undergone major surgical procedure 4 weeks prior to randomisation.

6. Subjects who are on anti-depressants, anti-psychotics.

7. Subjects with known history of clinically significant endocrine, gastrointestinal, cardiovascular, hematological, hepatic, immunological, renal, respiratory, or genitourinary abnormalities or diseases; except those that are considered etiology of said indication.

8. Females who are pregnant or nursing or planning to become pregnant during the study period.

### Study Design:

### An Open Label, Prospective, Randomised, Comparative, Two Arm Clinical Study

Investigational Product: Nichi Glucan

Comparator: None. Conventional Therapy only to be provided.

Indication: Covid19 caused by SARS-COV2 (beta COV)

Subject Population: Adult Subjects aged between 18 and 65 years (both ages all sexes inclusive) who are confirmed to be positive for SARS-COV2 by way of RT-PCR in a laboratory approved by MoH-FW and the State Government

Number of Subjects: 48 Subjects

Treatment Arms: Two

Treatment Arm I: Nichi Glucan+Conventional Therapy : 24 Subjects

Treatment Arm II: Conventional Therapy : 24 Subjects

Treatment Duration: Maximum of 30 days per enrolled Subject

### Assessments

1. Covid19 Clinical Symptoms: Day 1, Day 15, Day 30

2. RT PCR: Day 1, Day 15, Day 30

3. Immunological Parameters: Day 1, Day 15, Day 30

4. Hospitalisation Parameters: Day 1, Day 15, Day 30

5. Blood Parameters: Day 1, Day 15, Day 30

6. Chest Scan: Day 1, Day 15, Day 30

7. Quality of Life Questionnaire: Day 15, Day 30

Immunology: CD4, CD8, CD56, CD13, IgA, IL6

Hospitalisation: Mortality, ICU admission, Oxygen/LifeSupport

Blood Test: D-Dimer, CRP, ESR, FBG

Chest Scan: CT Lung

QoL Questionnaire

### References on Effects of Nichi Glucan AFO-202 in COVID-19

1. Decrease D-Dimer values—Ref: D-dimer level is associated with the severity of COVID-19. Thromb Res. 2020 November; 195:219-225.

2. Decrease IL-6 levels—Ref: Prognostic value of interleukin-6, C-reactive protein, and procalcitonin in patients with COVID-19. J Clin Virol. 2020 June; 127:104370.

3. Mounting anti-viral defense by upregulating NK cells and macrophages. Ref: Natural Killer Cell Dysfunction and Its Role in COVID-19. Int J Mol Sci. 2020 Sep. 1; 21(17):6351. doi: 10.3390/ijms21176351.

4. Increase virus-specific antibodies (IgG, IgM and sIgA) for neutralization of virus toxicity. Ref: Serum IgA, IgM, and IgG responses in COVID-19. Cell Mol Immunol. 2020 July; 17(7):773-775. doi: 10.1038/s41423-020-0474-z.

5. Helping in maintenance of blood glucose and lipid levels thereby preventing risk of severe COVID-19 due to comorbidities such as diabetes mellitus, dyslipidemia and obesity. Ref: Commentary: Beyond “TRIM” Benefits of β-Glucan by Blood Glucose and Lipid Balancing Potentials in Its Defense Against COVID-19. Front Immunol. 2021 Mar. 29; 12:620658. doi: 10.3389/fimmu.2021.620658

### Common Reference:

Immunological actions of Sophy beta-glucan (beta-1,3-1,6 glucan), currently available commercially as a health food supplement. Microbiol Immunol. 2007; 51(9):861-73. doi: 10.1111/j.1348-0421.2007.tb03982.x.

### Preliminary Outcome of Nichi Glucan AFO-202 in COVID-19

1. Hospital stay: Shorter hospital stay by average 5 days compared to the control Literature evidence on hospital stay after COVID: https://www.ncbi.nlm.nih.gov/pmc/articles/PMC7467845/)

2. IL-6 levels : IL6 levels decreased by 10 to 12% compared to control IL6 levels in COVID-19 patients : 1.85 pg/ml to 21.55 pg/ml Cut off value: 37.65 pg/ml https://translational-medicine.biomedcentral.com/articles/10.1186/s12967-020-02571-x

3. Anti-viral defense (by upregulating lymphocytes): Lymphocyte cell count increased by 1.35 folds

Lower Lymphocyte levels leads to increased COVID mortality—https://ehoonline.biomedcentral.com/articles/10.1186/s40164-021-00199-1

4. Virus-specific antibody levels: (IgG, IgM and sIgA) : Increase in antibody levels by 5 folds

Antibodies levels in COVID-19 patients—https://www.nature.com/articles/s41423-020-0474-z

5. Blood glucose and lipid levels balancing : In AFO-202 Nichi Glucan group the fasting blood glucose levels decreased from 4 to 21% decrease; Average Hba1C of 8.1 was either maintained or there was a decrease to 7.7

In control group, the fasting blood glucose levels increased from baseline by 15%; Lipid levels increased from 18 to 27%. Hba1C levels increased upto 8.5%. https://pubmed.ncbi.nlm.nih.gov/33051331/

6. D-Dimer control: D-Dimer levels decreased to normal values of less than 0.5 mirco FEG/ml while in control D-Dimer values remain increased above normal range https://www.ncbi.nlm.nih.gov/pmc/articles/PMC7384402/

7. Time taken RT PCR to become negative: There was a reduction in time taken for RT-PCR negativity by 2.4 days compared to control

Average time taken for RT-PCR to become negative—10 to 14 days https://bmcmedicine.biomedcentral.com/articles/10.1186/s12916-020-01810-8

### Conclusion

Proposed mechanisms for multi-organ dysfunction in COVID-19 are multi-factorial and a hypercoagulable state with micro and macro-circulatory thrombosis has been identified as a key factor deciding the clinical course and disease severity. D-dimer and prothrombin have emerged as the most important biomarkers to be analysed at the time of hospital admission due to COVID-19. Ethnically vulnerable populations such as Caucasians, African-Americans, the elderly and patients with co-morbidities form the population at high risk who need prevention of development of this hypercoagulable state. COVID-19 for which no pharmacological strategies for prevention or treatment are presently available preventive strategies including biological response modifiers for enhancing immunity and decreasing the risk of coagulopathy may be highly beneficial to combat the disease, especially in these vulnerable populations.

Beta glucans employ the majority of the components of the reticulo-endothelial system by inducing gut mucosal immunity, travelling to distant effector sites (such as the spleen and lymph nodes) and generating a “central” immunity memory in the bone marrow via TRIM—thus activating all aspects of the immune system [B4, 8-12] and resulting in a continuous, lasting immune response against various pathogens that can elicit specific antiviral immunity [B24]. Above all, this immune response has had a track record of safe consumption by all ages for over two decades [B24-27] apart from having been earlier employed as vaccine adjuvants.

Thus, without definitive therapeutics for COVID-19 and with significant hurdles in identifying an ideal vaccine with a wide-spectrum activity and no side effects, orally consumed beta glucans (such as the AFO-202 beta glucan) will serve as a wide-spectrum immune-balancing food-supplement-based enteric (β-WIFE) vaccine approach to COVID-19.

Several factors and pathogenic processes have been identified that can predispose an individual to a high risk of developing cancer and/or enable the progression of cancer: i. chronic- and micro-inflammation caused by infections, aging or metabolic disorders such as diabetes, ii. genetic causes, and iii. immune system weakness, either due to cancer or cancer therapy. Therefore, prevention of cancer in the general population and its spread in those undergoing surgical or chemotherapeutic treatments is practically feasible only if a consistent and simple approach can be followed, such as a nutritional supplement to combat immune system compromise and chronic microinflammation. In this review, we have presented the evidence of the BRM glucan for its potential function as a beta-glucan VACCIEN-adjuvant approach to conquer cancer through immunoenhancement. The B-VACCIEN approach may help to tackle cancer in specific immunocompromised populations, as it encompasses a wide variety of biological response modification in terms of balancing metabolic parameters such as blood glucose and lipid levels, apart from increasing peripheral blood cell cytotoxicity against cancer and alleviating chemotherapy side effects in animal models. Thus, we suggest this B-VACCIEN approach as a potential strategy for a long-term prophylaxis in people with such specific conditions of immunocompromise or those who are genetically prone to cancer.

### Modifications and other Embodiments

Various modifications and variations of the described glucan products, compositions and methods as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the chemical, biological, medical, environmental, cosmetic or food arts or related fields are intended to be within the scope of the following claims.

### REFERENCES

A1. Wang, C., Horby, P. W., Hayden, F. G., Gao, G. F. (2020). A novel coronavirus outbreak of global health concern. Lancet 395, 470-473.
A2. Lu, R., Zhao, X., Li, J., Niu, P., Yang, B., Wu, H. (2020). Genomic characterisation and epidemiology of 2019 novel coronavirus: implications for virus origins and receptor binding. Lancet 395, 565-574.
A3. World Health Organization. Coronavirus (COVID-19). https://covid19.who.int/(Accessed May 5, 2020).
A4. Chakraborty, C., Sharma, A. R., Sharma, G., Bhattacharya, M., Lee, S. S. (2020). SARS-CoV-2 causing pneumonia-associated respiratory disorder (COVID-19): diagnostic and proposed therapeutic options. Eur Rev Med Pharmacol Sci. 24, 4016-4026.
A5. Centre for Evidence-Based Medicine. Global Covid-19 Case Fatality Rates. https://www.cebm.net/covid-19/global-covid-19-case-fatality-rates/ (Accessed, May 5, 2020).
A6. Singh, A. K., Gupta, R., Misra, A. (2020). Comorbidities in COVID-19: Outcomes in hypertensive cohort and controversies with renin angiotensin system blockers. Diabetes Metab Syndr. 14, 283-287.
A7. Pedersen, F., Ho, Y. C. (2020). SARS-CoV-2: a storm is raging. J. Clin. Invest. 2020. doi: 10.1172/JCI137647.
A8. L,i G., Fan, Y., Lai, Y., Han, T., Li, Z., Zhou, P. (2020). Coronavirus infections and immune responses. J. Med. Virol. 92, 424-432.
A9. Cao, X. (2020). COVID-19: immunopathology and its implications for therapy. Nat Rev Immunol. 20, 269-270.
A10. Lagunas-Rangel, F. A. (2020). Neutrophil-to-lymphocyte ratio and lymphocyte-to-C-reactive protein ratio in patients with severe coronavirus disease 2019 (COVID-19): A meta-analysis. J Med Virol. doi: 10.1002/jmv.25819.
A11. Sun, X., Wang, T., Cai, D., Hu, Z., Chen, J., Liao, H., Zhi, L., Wei, H., Zhang, Z., Qiu, Y., Wang, J., & Wang, A. (2020). Cytokine storm intervention in the early stages of COVID-19 pneumonia. Cytokine & growth factor reviews, S1359-6101(20)30048-4. Advance online publication. https://doi.org/10.1016/j.cytogfr.2020.04.002
A12. Jamilloux, Y., Henry, T., Belot, A., Viel, S., Fauter, M., El Jammal, T., Walzer, T., Francois, B., & Seve, P. (2020). Should we stimulate or suppress immune responses in COVID-19? Cytokine and anti-cytokine interventions. Autoimmunity reviews, 102567. Advance online publication. https://doi.org/10.1016/j.autrev.2020.102567.
A13. Perricone, C., Triggianese, P., Bartoloni, E., Cafaro, G., Bonifacio, A. F., Bursi, R., et al. (2020). The anti-viral facet of anti-rheumatic drugs: Lessons from COVID-19. J Autoimmun. 102468. doi: 10.1016/j.jaut.2020.102468
A14. Tan, W., Aboulhosn, J. (2020). The cardiovascular burden of coronavirus disease 2019 (COVID-19) with a focus on congenital heart disease. Int J Cardiol. 309, 70-77.
A15. Clarke, R., Valdes-Marquez, E., Hill, M., Gordon, J., Farrall, M., Hamsten, A., et al. (2018). Plasma cytokines and risk of coronary heart disease in the PROCARDIS study. Open Heart. 5, e000807.
A16. Williams, J. W., Huang, L. H., Randolph, G. J. (2019). Cytokine Circuits in Cardiovascular Disease. Immunity. 50, 941-954.
A17. Fang, L., Karakiulakis, G., Roth, M. (2020). Are patients with hypertension and diabetes mellitus at increased risk for COVID-19 infection?. Lancet Respir Med. 8,e21.
A18. Muniyappa, R., Gubbi, S. (2020). COVID-19 pandemic, coronaviruses, and diabetes mellitus. Am J Physiol Endocrinol Metab. 318, E736-E741.
A19. Maddaloni, E., Buzzetti, R. (2020). Covid-19 and diabetes mellitus: unveiling the interaction of two pandemics. Diabetes Metab Res Rev. e33213321. doi: 10.1002/dma.3321.
A20. Ferrey, A. J., Choi, G., Hanna, R. M., Chang, Y., Tantisattamo, E., Ivaturi, K., et al. (2020). A Case of Novel Coronavirus Disease 19 in a Chronic Hemodialysis Patient Presenting with Gastroenteritis and Developing Severe Pulmonary Disease. Am J Nephrol. 1-6. doi: 10.1159/000507417.
A21. Pertosa, G., Grandaliano, G., Gesualdo, L., Schena, F. P. (2000). Clinical relevance of cytokine production in hemodialysis. Kidney Int Suppl. 76, S104-S111
A22. Jofre, R., Rodriguez-Benitez, P., Lopez-Gomez, J. M., Perez-Garcia, R. (2006). Inflammatory syndrome in patients on hemodialysis. J Am Soc Nephrol. 17, S274-S280.
A23. Crepin, T., Legendre, M., Carron, C., Vachey, C., Courivaud, C., Rebibou, J. M., et al. (2020). Uraemia-induced immune senescence and clinical outcomes in chronic kidney disease patients. Nephrol Dial Transplant. 35, 624-632.
A24. Liang, W., Guan, W., Chen, R., Wang, W., Li, J., Xu, K. (2020). Cancer patients in SARS-CoV-2 infection: a nationwide analysis in China. Lancet Oncol. 21, 335-337.
A25. Wang, H., and Zhang, L. (2020). Risk of COVID-19 for patients with cancer. Lancet Oncol. 21, e181.
A26. Guillen, E., Pineiro, G. J., Revuelta, I., Rodriguez, D., Bodro, M., Moreno, A., et al. (2020). Case report of COVID-19 in a kidney transplant recipient: Does immunosuppression alter the clinical presentation? Am J Transplant. doi: 10.1111/ajt.15874.
A27. Nair, V., Jandovitz, N., Hirsch, J. S., Nair, G., Abate, M., Bhaskaran, M., et al (2020). COVID-19 in kidney transplant recipients. Am J Transplant. doi: 10.1111/ajt.15967.
A28. Russell, B., Moss, C., George, G., Santaolalla, A., Cope, A., Papa, S., Van Hemelrijck, M. (2020). Associations between immune-suppressive and stimulating drugs and novel COVID-19-a systematic review of current evidence. Ecancermedcalscience, 14, 1022.
A29. Seminari, E., Colaneri, M., Sambo, M., Gallazzi, I., Di Matteo, A., Silvia, R., et al (2020). SARS Cov2 infection in a renal transplanted patients. A case report. Am J Transplant. doi: 10.1111/ajt.15902.
A30. Prompetchara, E., Ketloy, C., Palaga, T. (2020). Immune responses in COVID-19 and potential vaccines: Lessons learned from SARS and MERS epidemic. Asian Pacific journal of allergy and immunology, 38, 1-9.
A31. Patel, N., Penkert, R. R., Jones, B. G., Sealy, R. E., Surman, S. L., Sun, Y., et al (2019). Baseline Serum Vitamin A and D Levels Determine Benefit of Oral Vitamin A&D Supplements to Humoral Immune Responses Following Pediatric Influenza Vaccination. Viruses, 11, 907.
A32. Jayawardena, R., Sooriyaarachchi, P., Chourdakis, M., Jeewandara, C., Ranasinghe, P. (2020). Enhancing immunity in viral infections, with special emphasis on COVID-19: A review. Diabetes Metab Syndr. 14, 367-382.
A33. Girodon, F. (1999). Impact of trace elements and vitamin supplementation on immunity and infections in institutionalized elderly patients: a randomized controlled trial. Arch Intern Med. 159, 748-754.
A34. Graat, J. M., Schouten, E. G., Kok, F. J. (2002). Effect of daily vitamin E and multivitamin-mineral supplementation on acute respiratory tract infections in elderly Persons-A randomized controlled trial. JAMA. 288, 715-721.
A35. Akramiene, D., Kondrotas, A., Didziapetriene, J., & Kevelaitis, E. (2007). Effects of beta-glucans on the immune system. Medicina (Kaunas, Lithuania), 43(8), 597-606.
A36. Stier, H., Ebbeskotte, V., & Gruenwald, J. (2014). Immune-modulatory effects of dietary Yeast Beta-1,3/1,6-D-glucan. Nutrition journal, 13, 38. https://doi.org/10.1186/1475-2891-13-38.
A37. Vetvicka, V. & Vetvickova, J. (2014). Comparison of immunological effects of commercially available β-glucans. Appl Sci Rep. 1, 2. http://dx.doi.org/10.7243/2054-9903-1-2
A38. Vetvicka, V. & Vetvickova, J. (2016). Comparison of immunological effects of commercially available beta-glucans: part III. Int Clin Pathol J. 2, 78-83.
A39. Vetvicka, V. & Vetvickova, J. (2018). Glucans and Cancer: Comparison of Commercially Available β-glucans—Part IV. Anticancer Res. 38, 1327-1333.
A40. Bergendiova, K., Tibenska, E., & Majtan, J. (2011). Pleuran (β-glucan from Pleurotus ostreatus) supplementation, cellular immune response and respiratory tract infections in athletes. European journal of applied physiology, 111(9), 2033-2040.
A41. Dedeepiya, V. D., Sivaraman, G., Venkatesh, A. P., Preethy, S., Abraham, S. J. (2012). Potential effects of nichi glucan as a food supplement for diabetes mellitus and hyperlipidemia: preliminary findings from the study on three patients from India. Case Rep Med. 2012, 895370.
A42. Ganesh, J. S., Rao, Y. Y., Ravikumar, R., Jayakrishnan, G. A., Iwasaki, M., Preethy, S., et al (2014). Beneficial effects of black yeast derived 1-3, 1-6 Beta Glucan-Nichi Glucan in a dyslipidemic individual of Indian origin—a case report. J Diet Suppl. 11, 1-6.
A43. Ikewaki, N., Fujii, N., Onaka, T., Ikewaki, S., Inoko, H. (2007). Immunological actions of Sophy beta-glucan (beta-1,3-1,6 glucan), currently available commercially as a health food supplement. Microbiol Immunol. 51, 861-873.
A44. Brown, G. D., & Gordon, S. (2003). Fungal beta-glucans and mammalian immunity. Immunity. 19, 311-315.
A45. Cramer, D. E., Allendorf, D. J., Baran, J. T., Hansen, R., Marroquin, J., Li, B., et al. (2006). Beta-glucan enhances complement-mediated hematopoietic recovery after bone marrow injury. Blood. 107, 835-840.
A46. Ohno, N., Furukawa, M., Miura, N. N., Adachi, Y., Motoi, M., & Yadomae, T. (2001). Antitumor beta glucan from the cultured fruit body of Agaricus blazei. Biol. Pharm. Bull. 24, 820-828.
A47. https://www.betaglucan.org/fdagras/
A48. http://sophy-inc.co.jp/A49.
A49. Muramatsu, D., Iwai, A., Aoki, S., Uchiyama, H., Kawata, K., Nakayama, Y., et al. (2012). β-Glucan derived from Aureobasidium pullulans is effective for the prevention of influenza in mice. PLoS One.7, e41399.
A50. Yatawara, L., Wickramasinghe, S., Nagataki, M., Takamoto, M., Nomura, H., Ikeue, Y., et al. (2009). Aureobasidium-derived soluble branched (1,3-1,6) beta-glucan (Sophy beta-glucan) enhances natural killer activity in Leishmania amazonensis-infected mice. Korean J Parasitol. 47, 345-351.
A51. Tang, X. Y., Gao, J. S., Yuan, F., Zhang, W. X., Shao, Y. J., Sakurai, F., et al (2011). Effects of Sophy β-glucan on growth performance, carcass traits, meat composition, and immunological responses of Peking ducks. Poult Sci. 90, 737-745.
A52. Le, T., Le, T., Doan, T. H., Quyen, D., Le, K. X., Pham, V., et al. (2011). The adjuvant effect of Sophy β-glucan to the antibody response in poultry immunized by the avian influenza A H5N1 and H5N2 vaccines. J Microbiol Biotechnol. 21, 405-411.
A53. 28th Annual Meeting of Japanese Society for Parenteral and Enteral Nutrition (2013). Abstract
A54. Vetvicka, V., & Vetvickova, J. (2015). Glucan supplementation enhances the immune response against an influenza challenge in mice. Ann Transl Med. 3, 22.
A55. Muramatsu, D., Iwai, A., Aoki, S., Uchiyama, H., Kawata, K., Nakayama, Y., et al (2012). β-Glucan derived from Aureobasidium pullulans is effective for the prevention of influenza in mice. PLoS One. 7, e41399.
A56. Dharsono, T., Rudnicka, K., Wilhelm, M., & Schoen, C. (2019). Effects of Yeast (1,3)-(1,6)-Beta-Glucan on Severity of Upper Respiratory Tract Infections: A Double-Blind, Randomized, Placebo-Controlled Study in Healthy Subjects. J Am Coll Nutr. 38, 40-50.
A57. Elson, C. O., & Alexander, K. L. (2015). Host-microbiota interactions in the intestine. Dig Dis. 33, 131-136.
A58. Wang, Y., Ames, N. P., Tun, H. M., Tosh, S. M., Jones, P. J., Khafipour, E. (2016). High Molecular Weight Barley β-Glucan Alters Gut Microbiota Toward Reduced Cardiovascular Disease Risk. Front Microbiol. 7, 129.
A59. Raa J. (2015). Immune modulation by non-digestible and non-absorbable beta-1,3/1,6-glucan, Microbial Ecology in Health and Disease. 26, 27824.
A60. Nguyen, A., David, J. K., Maden, S. K., Wood, M. A., Weeder, B. R., Nellore, A., & Thompson, R. F. (2020). Human leukocyte antigen susceptibility map for SARS-CoV-2. Journal of virology, JVI.00510-20. Advance online publication. https://doi.org/10.1128/JVI.00510-20.
A61. Eric W. Stawiski, Devan Diwanji, Kushal Suryamohan, Ravi Gupta, Frederic A. Fellouse, J. Fah Sathirapongsasuti, Jiang Liu, et al (2020). Human ACE2 receptor polymorphisms predict SARS-CoV-2 susceptibilitybioRxiv 2020.04.07.024752; doi: https://doi.org/10.1101/2020.04.07.024752 Available at: https://www.biorxiv.org/content/10.1101/2020.04.07.024752v1 (Accessed Jun. 4, 2020).
A62. Zabetakis, I., Lordan, R., Norton, C., & Tsoupras, A. (2020). COVID-19: The Inflammation Link and the Role of Nutrition in Potential Mitigation. Nutrients, 12(5), E1466. https://doi.org/10.3390/nu12051466
A63. Rajarshi, K.; Chatterjee, A.; Ray, S. BCG Vaccination Strategy for Preventaion against COVID-19: Hype or Hope?. Preprints 2020, 2020040351. doi: 10.20944/preprints202004.0351.v1. Available at: https://www.preprints.org/manuscript/202004.0351/v1 (Accessed Jun. 4, 2020)
A64. Katoh, S., Obayashi, T., Ganesh, J. S., Iwasaki, M., Preethy, S., & Abraham, S. J. (2020). Cross-Protection Induced by Encephalitis Vaccines against COVID-19 Might be a Reason for Relatively Lower Mortality Rate in Some Countries. Archives of academic emergency medicine, 8(1), e54.
A65. Cascella, M., Rajnik, M., Cuomo, A., Dulebohn, S. C., & Di Napoli, R. (2020). Features, Evaluation and Treatment Coronavirus (COVID-19). In StatPearls. StatPearls Publishing.
A66. Fara, M. G., Stein, L. K., Skliut, M., Morgello, S., Fifi, J. T., & Dhamoon, M. S. (2020). Macrothrombosis and stroke in patients with mild Covid-19 infection. Journal of thrombosis and haemostasis : JTH, 10.1111/jth.14938.
a1. Nahum J, Morichau-Beauchant T, Daviaud F, Echegut P, Fichet J, Maillet J M, Thierry S. Venous Thrombosis Among Critically Ill Patients With Coronavirus Disease 2019 (COVID-19). JAMA Netw Open. 2020 May 1; 3(5):e2010478. doi: 10.1001/jamanetworkopen.2020.10478.
a2. Quartuccio L, Semerano L, Benucci M, Boissier M C, De Vita S. Urgent avenues in the treatment of COVID-19: Targeting downstream inflammation to prevent catastrophic syndrome. Joint Bone Spine. 2020;87(3):191-193. doi:10.1016/j.jbspin.2020.03.011
a3. Tufan A, Avanoglu Guler A, Matucci-Cerinic M. COVID-19, immune system response, hyperinflammation and repurposing antirheumatic drugs. Turk J Med Sci. 2020 Apr. 21; 50(SI-1):620-632. doi: 10.3906/sag-2004-168. PMID: 32299202; PMCID: PMC7195984.
a4. Fara M G, Stein L K, Skliut M, Morgello S, Fifi J T, Dhamoon M S. Macrothrombosis and stroke in patients with mild Covid-19 infection. J Thromb Haemost. 2020 May 28. doi: 10.1111/jth.14938. Epub ahead of print. PMID: 32464707.
a5. Fang L, Karakiulakis G, Roth M. Are patients with hypertension and diabetes mellitus at increased risk for COVID-19 infection? Lancet Respir Med. 2020 April; 8(4):e21. doi: 10.1016/S2213-2600(20)30116-8. Epub 2020 Mar. 11. Erratum in: Lancet Respir Med. 2020 May 18;: PMID: 32171062; PMCID: PMC7118626.
a6. Wang B, Li R, Lu Z, Huang Y. Does comorbidity increase the risk of patients with COVID-19: evidence from meta-analysis. Aging (Albany NY). 2020; 12(7):6049-6057
a7. Monteil V, Kwon H, Prado P, et al. Inhibition of SARS-CoV-2 Infections in Engineered Human Tissues Using Clinical-Grade Soluble Human ACE2. Cell. 2020; 181(4):905-913.e7. doi:10.1016/j.ce11.2020.04.004
a8. Iba T, Levy J H, Levi M, Connors J M, Thachil J. Coagulopathy of Coronavirus Disease 2019 [published online ahead of print, 2020 May 27]. Crit Care Med.
a9. Marchandot B, Sattler L, Jesel L, et al. COVID-19 Related Coagulopathy: A Distinct Entity?. J Clin Med. 2020; 9(6):E1651.
a10. Guan, W. J.; Ni, Z. Y.; Hu, Y.; Liang, W. H.; Ou, C. Q.; He, J. X.; Liu, L.; Shan, H.; Lei, C. L.; Hui, D. S.; et al. China Medical Treatment Expert Group for Covid-19. Clinical Characteristics of Coronavirus Disease 2019 in China. N. Engl. J. Med. 2020, 382, 1708-1720
a11. Tang N, Li D, Wang X, Sun Z. Abnormal coagulation parameters are associated with poor prognosis in patients with novel coronavirus pneumonia. J Thromb Haemost. 2020. doi:10.1111/jth.14768
a12. Zhou F, Yu T, Du R, et al. Clinical course and risk factors for mortality of adult inpatients with COVID19 in Wuhan, China: a retrospective cohort study. Lancet. 2020; 395(10229):1054-1062. doi:10.1016/50140-6736(20)30566-32020; 10.1097/CCM.0000000000004458. doi:10.1097/CCM.0000000000004458
a13. Lillicrap D. Disseminated intravascular coagulation in patients with 2019-nCoV pneumonia. J Thromb Haemost. 2020; 18(4):786-787. doi:10.1111/jth.14781
a14. Klok F A, Kruip M J H A, van der Meer N J M, Arbous M S, Gommers D A M P J, Kant K M, Kaptein F H J, van Paassen J, Stals M A M, Huisman M V, Endeman H (2020) Incidence of thrombotic complications in critically ill ICU patients with COVID-19. Thromb Res. On-line April 10
a15. Becker R C. COVID-19 update: Covid-19-associated coagulopathy [published online ahead of print, 2020 May 15]. J Thromb Thrombolysis. 2020; 1-14. doi:10.1007/s11239-020-02134-3
a16. Leonard-Lorant I, Delabranche X, Severac F, et al. Acute Pulmonary Embolism in COVID-19 Patients on CT Angiography and Relationship to D-Dimer Levels [published online ahead of print, 2020 Apr. 23]. Radiology. 2020;201561. doi:10.1148/radio1.2020201561
a17. Oxley T J, Mocco J, Majidi S, et al. Large-Vessel Stroke as a Presenting Feature of Covid-19 in the Young. N Engl J Med. 2020; 382(20):e60. doi:10.1056/NEJMc2009787
a18. Zhou F, Yu T, Du R, et al. Clinical course and risk factors for mortality of adult inpatients with COVID-19 in Wuhan, China: a retrospective cohort study [published correction appears in Lancet. 2020 Mar. 28; 395(10229):1038] [published correction appears in Lancet. 2020 Mar. 28; 395(10229):1038]. Lancet. 2020; 395(10229):1054-1062. doi:10.1016/S0140-6736(20)30566-3
a19. Zhang L, Feng X, Zhang D, et al. Deep Vein Thrombosis in Hospitalized Patients with Coronavirus Disease 2019 (COVID-19) in Wuhan, China: Prevalence, Risk Factors, and Outcome [published online ahead of print, 2020 May 18]. Circulation.
a20. COVID-19 coagulopathy: an evolving story The Lancet Haematology. Editorial 2020 7(6) DOI:https://doi.org/10.1016/S2352-3026(20)30151-4
a21. Huang C, Wang Y, Li X, et al. Clinical features of patients infected with 2019 novel coronavirus in Wuhan, China. Lancet. 2020; 395(10223):497-506
a22. Liao S, Woulfe T, Hyder S, Merriman E, Simpson D, Chunilal S. Incidence of venous thromboembolism in different ethnic groups: a regional direct comparison study. J Thromb Haemost. 2014; 12(2):214-219
a23. White R H, Keenan C R. Effects of race and ethnicity on the incidence of venous thromboembolism. Thromb Res. 2009; 123 Suppl 4:S11-17
a24. Fogarty H, Townsend L, Ni Cheallaigh C, et al. More on COVID-19 coagulopathy in Caucasian patients [published online ahead of print, 2020 May 12]. Br J Haematol. 2020; 10.1111/bjh.16791. doi:10.1111/bjh.16791
a25. McGonagle D, O'Donnell J S, Sharif K, Emery P, Bridgewood C. Immune mechanisms of pulmonary intravascular coagulopathy in COVID-19 pneumonia. The Lancet Rheumatology. DOI:https://doi.org/10.1016/S2665-9913(20)30121-1
a26. Mueller A L, McNamara M S, Sinclair D A. Why does COVID-19 disproportionately affect older people?. Aging (Albany N.Y.). 2020; 12(10):9959-9981.
a27. Jose R J, Manuel A. COVID-19 cytokine storm: the interplay between inflammation and coagulation [published online ahead of print, 2020 Apr 27]. Lancet Respir Med. 2020; 52213-2600(20)30216-2. doi:10.1016/S2213-2600(20)30216-2
a28. Thachil J, Agarwal S. Understanding the COVID-19 coagulopathy spectrum [published online ahead of print, 2020 May 21]. Anaesthesia. 2020; 10.1111/anae.15141. doi:10.1111/anae.15141
a29. Hussain A, Bhowmik B, do Vale Moreira N C. COVID-19 and diabetes: Knowledge in progress. Diabetes Res Clin Pract. 2020; 162:108142.
a30. Cao W, Li T. COVID-19: towards understanding of pathogenesis. Cell Res. 2020; 30(5):367-369. doi:10.1038/s41422-020-0327-4
a31. Kollias A, Kyriakoulis K G, Dimakakos E, Poulakou G, Stergiou G S, Syrigos K. Thromboembolic risk and anticoagulant therapy in COVID-19 patients: emerging evidence and call for action. Br J Haematol. 2020; 189(5):846-847. doi:10.1111/bjh.16727
a32. Engstad C S, Engstad R E, Olsen J O, Osterud B. The effect of soluble beta-1,3-glucan and lipopolysaccharide on cytokine production and coagulation activation in whole blood. Int Immunopharmacol. 2002; 2(11):1585-1597. doi:10.1016/s1567-5769(02)00134-0
a33. https://www.semanticscholar.org/paper/The-Role-of-Yeast-Beta-Glucan-on-Blood-Coagulation-El-Kashoury-Fattah/6f68a9067831eebb0015c2961882d1e91a89e84f
a34. Ikewaki N, Fujii N, Onaka T, Ikewaki S, Inoko H. Immunological actions of Sophy beta-glucan (beta-1,3-1,6 glucan), currently available commercially as a health food supplement. Microbiol Immunol. 2007; 51(9):861-873. doi:10.1111/j.1348-0421.2007.tb03982.x
a35. Dedeepiya VD, Sivaraman G, Venkatesh A P, Preethy S, Abraham S J. Potential effects of nichi glucan as a food supplement for diabetes mellitus and hyperlipidemia: preliminary findings from the study on three patients from India. Case Rep Med. 2012; 2012:895370. doi:10.1155/2012/895370
a36. Ganesh J S, Rao Y Y, Ravikumar R, et al. Beneficial effects of black yeast derived 1-3, 1-6 Beta Glucan-Nichi Glucan in a dyslipidemic individual of Indian origin—a case report. J Diet Suppl. 2014; 11(1):1-6. doi:10.3109/19390211.2013.859211
B1. Burton, D. R., & Walker, L. M. Rational Vaccine Design in the Time of COVID-19. Cell host & microbe 27, 695-698 (2020).
B2. Koirala, A., Joo, Y. J., Khatami, A., Chiu, C., & Britton, P. N. Vaccines for COVID-19: The current state of play. Paediatric respiratory reviews, S1526-054, 30095-6 (2020).
B3. Koyama, T., Weeraratne, D., Snowdon, J. L., & Parida, L. Emergence of Drift Variants That May Affect COVID-19 Vaccine Development and Antibody Treatment. Pathogens (Basel, Switzerland) 9, 324 (2020).
B4. Beverley, P. C. L. Immunology of vaccination, British Medical Bulletin 62, 15-28 (2002).
B5. Vetvicka, V., Vannucci, L., Sima, P., & Richter, J. Beta Glucan: Supplement or Drug? From Laboratory to Clinical Trials. Molecules (Basel, Switzerland) 24, 1251 (2019).
B6. Geller, A., & Yan, J. Could the Induction of Trained Immunity by β-Glucan Serve as a Defense Against COVID-19?. Frontiers in immunology 11, 1782 (2020).
B7. Arts, R., et al. Immunometabolic Pathways in BCG-Induced Trained Immunity. Cell reports, 17, 2562-2571 (2016).
B8. Del Corno, M., Gessani, S., & Conti, L. Shaping the Innate Immune Response by Dietary Glucans: Any Role in the Control of Cancer?. Cancers 12, 155 (2020).
B9. Goodridge, H. S., Wolf, A. J., & Underhill, D. M. Beta-glucan recognition by the innate immune system. Immunological reviews 230, 38-50(2009).
B10. Zou, S., Duan, B., & Xu, X. Inhibition of tumor growth by β-glucans through promoting CD4+ T cell immunomodulation and neutrophil-killing in mice. Carbohydrate polymers 213, 370-381 (2019).
B11. Li, B., Cai, Y., Qi, C., Hansen, R., Ding, C., Mitchell, T. C., & Yan, J. Orally administered particulate beta-glucan modulates tumor-capturing dendritic cells and improves antitumor T-cell responses in cancer. Clinical cancer research: an official journal of the American Association for Cancer Research 16, 5153-5164(2010).
B12. Richter, J., Kral, V., Vetvicka, V., Rajnohova Dobiasova, L., Fernandez-Botran, R. Effect of β-glucan Supplementation on Levels of IgM, IgA, IgG and Its Subclasses IgG1, IgG2, IgG3, and IgG4 in Cancer Patients. Journal of Tumor 4, 469-473 (2016).
B13. Kirkcaldy, R. D., King, B. A., & Brooks, J. T. COVID-19 and Postinfection Immunity: Limited Evidence, Many Remaining Questions. JAMA (2020).
B14. Petit, J., & Wiegertjes, G. F. Long-lived effects of administering β-glucans: Indications for trained immunity in fish. Developmental and comparative immunology 64, 93-102 (2016).
B15. FDA GRAS. https://www.betaglucan.org/fdagras/
B16. Bashir, K., & Choi, J. S. Clinical and Physiological Perspectives of β-Glucans: The Past, Present, and Future. International journal of molecular sciences 18, 1906 (2017).
B17. Infusino, F., et al. Diet Supplementation, Probiotics, and Nutraceuticals in SARS-CoV-2 Infection: A Scoping Review. Nutrients 12, 1718 (2020).
B18. Jayachandran, M., Chen, J., Chung, S., & Xu, B. A critical review on the impacts of β-glucans on gut microbiota and human health. The Journal of nutritional biochemistry 61, 101-110 (2018).
B19. Talbott, S. M., & Talbott, J. A. Baker's yeast beta-glucan supplement reduces upper respiratory symptoms and improves mood state in stressed women. Journal of the American College of Nutrition 31, 295-300 (2012).
B20. Dharsono, T., Rudnicka, K., Wilhelm, M., & Schoen, C. Effects of Yeast (1,3)-(1,6)-Beta-Glucan on Severity of Upper Respiratory Tract Infections: A Double-Blind, Randomized, Placebo-Controlled Study in Healthy Subjects. Journal of the American College of Nutrition 38, 40-50 (2019).
B21. Fuller, R., et al. Yeast-derived β-1,3/1,6 glucan, upper respiratory tract infection and innate immunity in older adults. Nutrition (Burbank, Los Angeles County, Calif.) 39- 40, 30-35 (2017).
B22. Jesenak, M., Majtan, J., Rennerova, Z., Kyselovic, J., Banovcin, P., & Hrubisko, M. Immunomodulatory effect of pleuran (β-glucan from Pleurotus ostreatus) in children with recurrent respiratory tract infections. International immunopharmacology 15, 395-399 (2013).
B23. Murphy, E. J., Masterson, C., Rezoagli, E., O'Toole, D., Major, I., Stack, G. D., Lynch, M., Laffey, J. G., & Rowan, N. J. β-Glucan extracts from the same edible shiitake mushroom Lentinus edodes produce differential in-vitro immunomodulatory and pulmonary cytoprotective effects—Implications for coronavirus disease (COVID-19) immunotherapies. The Science of the total environment 732, 139330 (2020).
B24. Ikewaki, N., Fujii, N., Onaka, T., Ikewaki, S., & Inoko, H. Immunological actions of Sophy beta-glucan (beta-1,3-1,6 glucan), currently available commercially as a health food supplement. Microbiology and immunology, 51, 861-873 (2007).
B25. Rao, K. S., Suryaprakash, V., Senthilkumar, R., Preethy, S., Katoh, S., Ikewaki, N., & Abraham, S. Role of Immune Dysregulation in Increased Mortality Among a Specific Subset of COVID-19 Patients and Immune-Enhancement Strategies for Combatting Through Nutritional Supplements. Frontiers in immunology 11, 1548 (2020).
B26. Yatawara, L., et al. Aureobasidium-derived soluble branched (1,3-1,6) beta-glucan (Sophy beta-glucan) enhances natural killer activity in Leishmania amazonensis—infected mice. The Korean journal of parasitology, 47, 345-351 (2009).
B27. Ikewaki N. Results of oral consumption of AFO-202 Beta Glucan in elderly volunteers and cancer patients through NK cell activity. Abstract presented at 28th Annual Meeting of Japanese Society for Parenteral and Enteral Nutrition (2013).
B28. Dedeepiya, V. D., Sivaraman, G., Venkatesh, A. P., Preethy, S., & Abraham, S. J. Potential effects of nichi glucan as a food supplement for diabetes mellitus and hyperlipidemia: preliminary findings from the study on three patients from India. Case reports in medicine 2012, 895370 (2012).
B29. Ganesh, J. S., Rao, Y. Y., Ravikumar, R., Jayakrishnan, G. A., Iwasaki, M., Preethy, S., & Abraham, S. J. Beneficial effects of black yeast derived 1-3, 1-6 Beta Glucan-Nichi Glucan in a dyslipidemic individual of Indian origin—a case report. Journal of dietary supplements 11, 1-6 (2014).
B30. Pasetti, M. F., Simon, J. K., Sztein, M. B., & Levine, M. M. Immunology of gut mucosal vaccines. Immunological reviews 239, 125-148 (2011).
B31. Criscuolo, E., Caputo, V., Diotti, R. A., Sautto, G. A., Kirchenbaum, G. A., & Clementi, N. Alternative Methods of Vaccine Delivery: An Overview of Edible and Intradermal Vaccines. Journal of immunology research 2019, 8303648 (2019).
B32. Reticuloendothelial system. In: Moreland L. W. (eds) Rheumatology and Immunology Therapy. Springer, Berlin, Heidelberg (2004).
B33. Vetvicka V, Vannucci L, Sima P. β-glucan as a new tool in vaccine development. Scand J Immunol. 2020;91(2):e12833.
B34. Le T, Le T, Doan T H, et al. The adjuvant effect of Sophy β-glucan to the antibody response in poultry immunized by the avian influenza A H5N1 and H5N2 vaccines. J Microbiol Biotechnol. 2011; 21(4):405-411.
C1. World Health Organization (WHO) Fact sheet on cancer https://www.who.int/news-room/fact-sheets/detail/cancer#:˜:text=Cancer%20is%20the %20second%20leading,%2D%20and%20middle%2Dincome%20countries. [Accessed Feb. 16, 2021].
C2. Wang M, Zhao J, Zhang L, Wei F, Lian Y, Wu Y, Gong Z, Zhang S, Zhou J, Cao K, Li X, Xiong W, Li G, Zeng Z, Guo C. Role of tumor microenvironment in tumorigenesis. J Cancer (2017) 8:761-773.
C3. Gonzalez H, Hagerling C, Werb Z. Roles of the immune system in cancer: from tumor initiation to metastatic progression. Genes Dev (2018) 32:1267-1284.
C4. Hanahan D, Weinberg R A. Hallmarks of cancer: the next generation. Cell (2011) 144:646-74.
C5. Balkwill F, Mantovani A. Inflammation and cancer: back to Virchow? Lancet (2001) 357:539-45.
C6. Bosch F X, Lorincz A, Munoz N, Meijer C J, Shah K V. The causal relation between human papillomavirus and cervical cancer. J Clin Pathol (2002) 55:244-65.
C7. Dunn G P, Old L J, Schreiber R D. The three Es of cancer immunoediting. Annu Rev Immunol (2004) 22:329-60.
C8. Angell H, Galon J. From the immune contexture to the Immunoscore: the role of prognostic and predictive immune markers in cancer. Curr Opin Immunol (2013) 25: 61-7.
C9. Pandya P H, Murray M E, Pollok K E, Renbarger J L. The Immune System in Cancer Pathogenesis: Potential Therapeutic Approaches. J Immunol Res (2016) 2016:4273943.
C10. Wu Y, Liu Y, Dong Y, Vadgama J. Diabetes-associated dysregulated cytokines and cancer. Integr Cancer Sci Ther (2016) 3 :370-78.
C11. Giovannucci E, Harlan D M, Archer M C, Bergenstal R M, Gapstur S M, Habel L A, Pollak M, Regensteiner J G, Yee D. Diabetes and cancer: a consensus report. Diabetes Care (2010) 33: 1674-85.
C12. Pitocco D, Tesauro M, Alessandro R, Ghirlanda G, Cardillo C. Oxidative stress in diabetes: implications for vascular and other complications. Int J Mol Sci (2013) 14: 21525-21550.
C13. Chang S C, Yang W V. Hyperglycemia, tumorigenesis, and chronic inflammation. Crit Rev Oncol Hematol (2016) 108:146-153.
C14. Liefvendahl E, Arnqvist H J. Mitogenic effect of the insulin analogue glargine in malignant cells in comparison with insulin and IGF-I. Horm Metab Res (2008) 40: 369-374.
C15. Esposito K, Nappo F, Marfella R, Giugliano G, Giugliano F, Ciotola M, Quagliaro L, Ceriello A, Giugliano D. Inflammatory cytokine concentrations are acutely increased by hyperglycemia in humans: role of oxidative stress. Circulation (2002) 106: 2067-2072.
C16. Costantini S, Capone F, Guerriero E, Marfella R, Sorice A, et al Costantini S, Capone F, Guerriero E, Marfella R, Sorice A, Maio P, Di Stasio M, Paolisso G, Castello G, Colonna G. Cytokinome profile of patients with type 2 diabetes and/or chronic hepatitis C infection. PLoS One (2012) 7: e39486
C17. Cai D, Khor S. “Hypothalamic Microinflammation” Paradigm in Aging and Metabolic Diseases. Cell Metab (2019) 30:19-35.
C18. De Souza C T, Araujo E P, Bordin S, Ashimine R, Zollner R L, Boschero A C, Saad M J, Velloso L A. Consumption of a fat-rich diet activates a proinflammatory response and induces insulin resistance in the hypothalamus. Endocrinology (2005) 146:4192-9.
C19. The genetics of Cancer. https://www.cancer.gov/about-cancer/causes-prevention/genetics [Accessed Feb. 16, 2021]
C20. Palomero L, Galvan-Femenia I, de Cid R, Espin R, Barnes D R, Cimba, Blommaert E, Gil-Gil M, Falo C, Stradella A, Ouchi D, Roso-Llorach A, Violan C, Pena-Chilet M, Dopazo J, Extremera A I, Garcia-Valero M, Herranz C, Mateo F, Mereu E, Beesley J, Chenevix-Trench G, Roux C, Mak T, Brunet J, Hakem R, Gorrini C, Antoniou A C, Lazaro C, Pujana M A. Immune Cell Associations with Cancer Risk. iScience (2020) 23:101296.
C21. Kresovich J K, O'Brien K M, Xu Z, Weinberg C R, Sandler D P, Taylor J A. Prediagnostic Immune Cell Profiles and Breast Cancer. JAMA Netw Open. (2020); 3: e1919536.
C22. Margolis K L, Rodabough R J, Thomson C A, Lopez A M, McTiernan A; Women's Health Initiative Research Group. Prospective study of leukocyte count as a predictor of incident breast, colorectal, endometrial, and lung cancer and mortality in post-menopausal women. Arch Intern Med (2007) 167:1837-44.
C23. Imai K, Matsuyama S, Miyake S, Suga K, Nakachi K. Natural cytotoxic activity of peripheral-blood lymphocytes and cancer incidence: an 11-year follow-up study of a general population. Lancet. (2000) 356:1795-9.
C24. Litman G W, Cannon J P, Dishaw L J. Reconstructing immune phylogeny: new perspectives. Nat Rev Immunol. (2005) 5:866-79.
C25. Aktipis C A, Boddy A M, Jansen G, Hibner U, Hochberg M E, Maley C C, Wilkinson G S. Cancer across the tree of life: cooperation and cheating in multicellularity. Philos Trans R Soc Lond B Biol Sci. (2015) 370 :20140219.
C26. Brussow H. The not so universal tree of life or the place of viruses in the living world. Philos Trans R Soc Lond B Biol Sci. (2009) 364: 2263-74.
C27. Kang D H, Weaver M T, Park N J, Smith B, McArdle T, Carpenter J. Significant impairment in immune recovery after cancer treatment. Nurs Res (2009) 58:105-14.
C28. Coffey J C, Wang J H, Smith M J, Bouchier-Hayes D, Cotter T G, Redmond H P. Excisional surgery for cancer cure: therapy at a cost. Lancet Oncol (2003) 4:760-8.
C29. Angka L, Khan S T, Kilgour M K, Xu R, Kennedy M A, Auer R C. Dysfunctional Natural Killer Cells in the Aftermath of Cancer Surgery. Int J Mol Sci. (2017); 18:1787.
C30. Immunization: The Basics. https://www.cdc.gov/vaccines/vac-gen/imz-basics.htm [Accessed Feb. 16, 2021].
C31. Leonardi G C, Accardi G, Monastero R, Nicoletti F, Libra M. Ageing: from inflammation to cancer. Immun Ageing. (2018) 15:1
C32. Finn O J. Vaccines for cancer prevention: a practical and feasible approach to the cancer epidemic. Cancer Immunol Res (2014) 2:708-13.
C33. Melief C J, van Hall T, Arens R, Ossendorp F, van der Burg S H. Therapeutic cancer vaccines. J Clin Invest. 2015 (125):3401-12.
C34. Thomas S, Prendergast G C. Cancer Vaccines: A Brief Overview. Methods Mol Biol. (2016) 1403:755-61.
C35. Adjuvants: Introduction. British Society of immunology. https://www.immunology.org/public-information/bitesized-immunology/vaccines-and-therapeutics/adjuvants-introduction [Accessed Feb. 16, 2021].
C36. Hu H G, Li Y M. Emerging Adjuvants for Cancer Immunotherapy. Front. Chem (2020) 8:601.
C37. Crunkhorn S. Strengthening the sting of immunotherapy. Nat Rev Immunol (2020) 20:589.
C38. Akramiene D, Kondrotas A, Didziapetriene J, Kevelaitis E. Effects of beta-glucans on the immune system. Medicina (Kaunas) (2007) 43:597-606.
C39. Zhang M, Kim JA, Huang A Y. Optimizing Tumor Microenvironment for Cancer Immunotherapy: β-Glucan-Based Nanoparticles. Front Immunol. (2018) 9:341.
C40. Vetvicka V, Vannucci L, Sima P, Richter J. Beta Glucan: Supplement or Drug? From Laboratory to Clinical Trials. Molecules. (2019) 24:1251.
C41. Seya T, Takeda Y, Takashima K, Yoshida S, Azuma M, Matsumoto M. Adjuvant immunotherapy for cancer: both dendritic cell-priming and check-point inhibitor blockade are required for immunotherapy. Proc Jpn Acad Ser B Phys Biol Sci (2018) 94:153-160.
C42. Kalafati L, Kourtzelis I, Schulte-Schrepping J, Li X, Hatzioannou A, Grinenko T, Hagag E, Sinha A, Has C, Dietz S, de Jesus Domingues A M, Nati M, Sormendi S, Neuwirth A, Chatzigeorgiou A, Ziogas A, Lesche M, Dahl A, Henry I, Subramanian P, Wielockx B, Murray P, Mirtschink P, Chung K J, Schultze J L, Netea M G, Hajishengallis G, Verginis P, Mitroulis I, Chavakis T. Innate Immune Training of Granulopoiesis Promotes Anti-tumor Activity. Cell (2020) 183:771-785.
C43. Katoh S, Rao K S, Suryaprakash V, Horiguchi A, Kushibiki T, Ojima K, Iwasaki M, Takeda M, Senthilkumar R, Rajmohan M, Karthick R, Preethy S, Abraham S. β-Glucans: Wide-spectrum Immune-balancing Food-supplement-based Enteric (β-WIFE) Vaccine Adjuvant Approach to COVID-19. Human Vaccines & Immunotherapeutics (2021) 17.
C44. Geller A, Yan J. Could the Induction of Trained Immunity by β-Glucan Serve as a Defense Against COVID-19? Front Immunol (2020) 11:1782.
C45. Vetvicka V, Vetvickova J. Glucans and Cancer: Comparison of Commercially Available β-glucans—Part IV. Anticancer Res (2018) 38:1327-1333.
C46. Ikewaki N, Fujii N, Onaka T, Ikewaki S, Inoko H. Immunological actions of Sophy beta-glucan (beta-1,3-1,6 glucan), currently available commercially as a health food supplement. Microbiol Immunol (2007) 51:861-73
C47. Yano et al. Sophy Beta-Glucan is effective in alleviating increased blood sugar levels. Abstract presented at the 55th Conference of the Japanese Society of Nutrition and Dietetics 2008.
C48. Dedeepiya V D, Sivaraman G, Venkatesh A P, et al. Potential effects of nichi glucan as a food supplement for diabetes mellitus and hyperlipidemia: preliminary findings from the study on three patients from India. Case Rep Med 2012 (2012) :895370.
C49. Ganesh J S, Rao Y Y, Ravikumar R, Jayakrishnan G A, Iwasaki M, Preethy S, Abraham S J. Beneficial effects of black yeast derived 1-3, 1-6 Beta Glucan-Nichi Glucan in a dyslipidemic individual of Indian origin--a case report. J Diet Suppl (2014) 11:1-6.
C50. Yatawara L, Wickramasinghe S, Nagataki M, Takamoto M, Nomura H, Ikeue Y, Watanabe Y, Agatsuma T. Aureobasidium-derived soluble branched (1,3-1,6) beta-glucan (Sophy beta-glucan) enhances natural killer activity in Leishmania amazonensis-infected mice. Korean J Parasitol (2009) 47:345-51.
C51. Chiba S, Ikushima H, Ueki H, Yanai H, Kimura Y, Hangai S, Nishio J, Negishi H, Tamura T, Saijo S, Iwakura Y, Taniguchi T. Recognition of tumor cells by Dectin-1 orchestrates innate immune cells for anti-tumor responses. Elife (2014) 3: e04177.
C52. Le T, Le T, Doan T H, Quyen D, Le K X, Pham V, Nagataki M, Nomura H, Ikeue Y, Watanabe Y, Agatsuma T. The adjuvant effect of Sophy β-glucan to the antibody response in poultry immunized by the avian influenza A H5N1 and H5N2 vaccines. J Microbiol Biotechnol (2011) 21(4):405-11.
C53. Yamazaki K, Watanabe Y, Nagata S, Ikegami H, Fujii N, Kato S, Aizono H. Anti-tumor effects of orally administered beta-glucan from black yeast. Abstract presented at the Annual Meeting of the Japan Society of Agricultural Chemistry, Japan (2004).
C54. Mizobuchil S, Watanabe Y, Yano H, Sasaguri S, Watanabe Y, Yano H. Analysis of innate immune stimulating effect of Sofy β-glucan during chemotherapy. Abstract presented at the 108th Regular Academic Meeting of the Japanese Society of Surgery, Japan (2008).
C55. Ma L, Wang H, Wang C, Su J, Xie Q, Xu L, Yu Y, Liu S, Li S, Xu Y, Li Z. Failure of Elevating Calcium Induces Oxidative Stress Tolerance and Imparts Cisplatin Resistance in Ovarian Cancer Cells. Aging Dis (2016) 7:254-66.
C56. Mizobuchi S, Taniwaki C, Watanabe Y, Sasaguri S. Antitumor effects of combined therapy with intraperitoneal CDDP and oral Sofy β-glucan. Abstract presented at the 63rd General Meeting of the Japanese Society of Gastroenterology, Japan (2008).
C57. Okumura T. NK cell cytotoxicity in different age groups of healthy volunteers. Gendai kagaku (Chemistry today) (1984) 11: 40.
C58. Mio M. Effect of oral intake of black yeast beta-glucan on NK activity in the elderly and patients with cancer. Abstract presented at 29th Annual Meeting of the Japanese Society of Venous and Enteral Nutrition Pacifico Yokohama, Japan (2014).
C59. Konjevic G, Jurisic V, Jovic V, Vuletic A, Mirjacic Martinovic K, Radenkovic S, Spuzic I. Investigation of NK cell function and their modulation in different malignancies. Immunol Res (2012) 52:139-56.
C60. Papaioannou N E, Beniata O V, Vitsos P, Tsitsilonis O, Samara P. Harnessing the immune system to improve cancer therapy. Ann Transl Med (2016) 4:261.

1-10. (canceled)

11. A method of regulating immune response in a subject, comprising:
increasing the ratio of lymphocytes to C-reactive-protein (LCR) and decreasing the ratio of neutrophils to lymphocytes (NLR) in a subject, the increasing including orally administering an effective amount of beta-1,3-1,6 glucan produced by Aureobasidium pullulans;
decreasing C-reactive protein (CRP) levels in the subject, the decreasing including orally administering an effective amount of beta-1,3-1,6 glucan produced by Aureobasidium pullulans;
decreasing IL-6 levels in the subject, the decreasing including orally administering an effective amount of beta-1,3-1,6 glucan produced by Aureobasidium pullulans;
decreasing D-dimer in the subject, the decreasing including orally administering an effective amount of beta-1,3-1,6 glucan produced by Aureobasidium pullulans;
increasing IgA in the subject, the increasing including orally administering an effective amount of beta-1,3-1,6 glucan produced by Aureobasidium pullulans;
increasing IgM in the subject, the increasing including orally administering an effective amount of beta-1,3-1,6 glucan produced by Aureobasidium pullulans;
increasing CD11b integrin levels in the subject, the decreasing including orally administering an effective amount of beta-1,3-1,6 glucan produced by Aureobasidium pullulans;
increasing mitochondrial ATPase production and mitochondrial DNA (ND1) in the subject, the increasing including orally administering an effective amount of beta-1,3-1,6 glucan produced by Aureobasidium pullulans;
increasing anti-CD69 antibody in the subject, the increasing including orally administering an effective amount of beta-1,3-1,6 glucan produced by Aureobasidium pullulans;
increasing anti-candida antibody in the subject, the increasing including orally administering an effective amount of beta-1,3-1,6 glucan produced by Aureobasidium pullulans;
or a combination thereof.

12. The method of claim 11, wherein the regulating is used in a treatment and/or prevention of a cancer, the method comprising the decreasing IL-6 levels in the subject.

13. The method of claim 12, wherein the regulating is used in a treatment and/or prevention of a solid cancer,
the regulating including increasing T cell count and B cell count in the subject; and,
the increasing including orally administering an effective amount of beta-1,3-1,6 glucan produced by Aureobasidium pullulans.

14. The method of claim 13, wherein the solid cancer is a renal carcinoma.

15. The method of claim 12, wherein the regulating is used in a treatment of a liquid cancer,
administering a chemotherapy agent selected from the group consisting of rituximab, cyclophosphamide, doxorubicin, vincristine, and prednisone, and a combination thereof; and,
orally administering an effective amount of beta-1,3-1,6 glucan produced by Aureobasidium pullulans;
wherein, the nausea from the chemotherapy in the subject is alleviated.

16. The method of claim 15, wherein the liquid cancer is a lymphoma.

17. The method of claim 11, wherein the regulating is used in an inhibition of blood coagulation, the method comprising the decreasing D-dimer in the subject.

18. The method of claim 11, wherein the regulating is used in a prevention of a viral infection, the method comprising the increasing of IgA in the subject.

19. A method of at least inhibiting the onset of SARS-CoV2 infections in a subject, the method comprising:
orally administering an effective amount of beta-1,3-1,6 glucan produced by Aureobasidium pullulans to the subject.

20. The method of claim 19, wherein the orally administering results in a decreasing expression of angiotensin converting enzyme 2 (ACE2), CD13, PDL-1, or a combination thereof, in the subject.

21. The method of claim 20 including treating SARS-CoV2 infections in the subject, wherein the effective amount of the beta-1,3-1,6 glucan produced by Aureobasidium pullulans is administered as an adjuvant to a conventional COVID 19 therapy.
